(19) United States
(12) Patent Application Publication
(10) Pub. No.: US 2026/0139007 A1
(43) Pub. Date: May 21, 2026

(54) EPHA4 ANTAGONISTS AND USES THEREOF

(71) Applicant: SANFORD BURNHAM PREBYS MEDICAL DISCOVERY INSTITUTE, La Jolla, CA (US)

(72) Inventor: Elena B. PASQUALE, La Jolla, CA (US)

(21) Appl. No.: 19/376,629

(22) Filed: Oct. 31, 2025

Related U.S. Application Data
(63) Continuation of application No. PCT/US2024/027729, filed on May 3, 2024.
(60) Provisional application No. 63/500,431, filed on May 5, 2023.

(51) Int. Cl. C07K 14/00; A61K 38/00; A61P 25/02; A61P 25/28

(52) U.S. Cl. CPC C07K 14/001; A61P 25/02; A61P 25/28; A61K 38/00

(57) ABSTRACT

Described herein are EphA4 receptor antagonists, pharmaceutical compositions containing EphA4 antagonists, and methods and uses of treating an EphA4-based disease, disorder or pathology in an individual using EphA4 receptor antagonists.

### CROSS-REFERENCE

This application is a continuation application of PCT Application No. PCT/US2024/027729, filed on May 3, 2024 and published as WO 2024/233346 A1 on Nov. 14, 2024, which claims the benefit of U.S. Provisional Application No. 63/500,431 filed May 5, 2023. The content of each of these related applications is incorporated herein by reference in its entirety.

### STATEMENT AS TO FEDERALLY SPONSORED RESEARCH

This invention was made with government support under R01 AG062617 awarded by the National Institutes of Health (NIH). The government has certain rights in the invention.

### SEQUENCE LISTING

The instant application contains a Sequence Listing which has been submitted electronically in XML format and is hereby incorporated by reference in its entirety. Said XML copy, created on May 2, 2024, is named 96SB-705018-US_SequenceListing.xml and is 102,527 bytes in size.

### FIELD OF THE INVENTION

The Ephrin (Eph) receptors are a large family of receptor tyrosine kinases with many functions in physiology and disease. They bind their activating ligands, the ephrins, mainly through a high-affinity binding pocket located in the N-terminal ephrin-binding domain. Each of the five ephrin-A ligands can bind to most of the nine EphA receptors and each of the three ephrin-B ligands can bind to the five EphB receptors. A cysteine-rich region and two fibronectin type III domains connect the ephrin-binding domain to the transmembrane segment. The cytoplasmic portion of the Eph receptors includes a juxtamembrane segment, the kinase domain, a sterile-alpha-motif (SAM) domain and a C-terminal PDZ domain-binding motif. Interaction between Eph receptors and ephrin ligands, which are attached to the cell surface through a GPI-anchor (ephrin-As) or a transmembrane domain (ephrin-Bs), typically occurs at sites of cell-cell contact. Ephrin binding promotes activation of the receptor's kinase domain, triggering “forward” signals. Ephrin ligands engaged with Eph receptors can also affect the cells in which they are expressed by mediating “reverse” signals.

Ephrin type-A receptor 4 (EphA4) signaling can be activated by all ephrin ligands, including the five GPI-linked ephrin-As and the three transmembrane ephrin-Bs. Highly expressed in the nervous system, EphA4 tyrosine kinase activity and downstream signaling leads to inhibition of axon growth and retraction of synaptic structures known as dendritic spines. The repulsive effects of EphA4 in neurons help guide the growth of developing axons towards their synaptic targets and may contribute to inhibition of axon regeneration following injury. In addition, EphA4 interaction with the ephrin-A3 ligand expressed in astrocytes stimulates “reverse” signals through the ephrin that limit the uptake of the extracellular neurotransmitter glutamate, thus modulating synaptic transmission. EphA4 is also highly expressed in adult hippocampal neurons, where it controls synaptic morphology and plasticity. Furthermore, EphA4 appears to contribute to the maintenance of brain neural stem cells in an undifferentiated state. This is in contrast to muscle, where EphA4 may contribute to myoblast differentiation.

Dysregulation of EphA4 activity and/or function has been implicated in the pathophysiology of neurodegenerative disorders, the promotion of neurotoxicity, the inhibition of nerve differentiation and regeneration, and in the progression of cancer. For example, low EphA4 expression and loss-of-function mutations are linked to late onset and prolonged survival in amyotrophic lateral sclerosis (ALS), a fatal disease that still lack any means for effective therapeutic intervention. Even partial EphA4 gene inactivation has shown beneficial effects in animal models of ALS, making EphA4 inhibition an attractive strategy for counteracting neurodegeneration. In addition, EphA4 was identified as a possible inhibitor of nerve regeneration after spinal cord injury. Experiments in mice suggest a role for EphA4 in the behavioral responses to cocaine administration. Further evidence also supports the involvement of EphA4 in the pathogenesis of spinal cord injury and other neurological diseases such as Alzheimer's disease, multiple sclerosis, stroke and traumatic brain injury. These pathological roles of EphA4 in the diseased nervous system are regarded as being linked to its increased expression and activation by ephrin ligands or Aβ-oligomers in the Alzheimer's brain, leading to abnormal inhibition of axon growth, synaptic function and neuronal survival. Furthermore, EphA4 signaling prevents the generation of cochlear sensory hair cells suggesting that inhibition of EphA4 activity could be an effective therapy in the treatment of hearing loss. Finally, increasing evidence also implicates EphA4 in various types of cancer, including glioblastoma, gastric cancer, pancreatic cancer, prostate cancer and breast cancer. For example, EphA4 downregulation studies have suggested a role for EphA4 in leukemia, prostate cancer, pancreatic cancer and gastric cancer cell growth and in liver cancer metastasis. High EphA4 expression has also been correlated with shorter survival in breast and gastric cancer patients, although the opposite correlation was found in lung cancer patients. EphA4 is also highly upregulated in Sezary syndrome, a leukemic variant of cutaneous T-cell lymphomas. Finally, EphA4 can enhance the oncogenic effects of fibroblast growth factor receptor 1 in glioblastoma cells. Hence, inhibiting EphA4-ephrin interaction could be useful for promoting axon regeneration and neural repair, providing neuroprotection and regulating synaptic plasticity in the nervous system as well as inhibiting the progression of cancer.

The two main strategies to block ephrin-induced EphA4 receptor signaling are inhibition of EphA4 kinase activity using kinase inhibitors and inhibition of ephrin binding to the EphA4 ligand binding domain using antagonists. Kinase inhibitors are hampered by low selectivity because they typically target multiple kinases due to the high conservation of the ATP binding pocket. As such, it is very difficult to identify kinase inhibitors selective for EphA4. In contrast, the ephrin-binding pocket in the extracellular EphA4 ligand binding domain has unique features that can be exploited for more selective antagonist targeting. However, the ephrin-binding pocket is very broad (exceeding 900 Å²) and shallow for high affinity binding of small molecules, and small molecule EphA4 antagonists found to date are not very potent and exhibit problematic features that make them unsuitable for therapeutic applications. On the other hand, peptide antagonists have been identified that are highly selective for the ephrin-binding pocket of EphA4. The most potent peptide antagonist identified by phage display was the linear dodecapeptide KYL (CAS Registry Number: 676657-00-4), which was shown to specifically inhibit EphA4 signaling in culture systems and animal models. The KYL peptide significantly dampened ALS pathogenesis in the classic rat SOD1 G93A ALS model. In addition, recent data have shown that KYL peptide can inhibit the toxic effects of Aβ oligomers in in vitro and in vivo mouse models of Alzheimer's disease. The KYL peptide was also shown to promote axon sprouting and recovery of limb function in a rat model of spinal cord injury. Thus, the KYL peptide clearly demonstrated the therapeutic potential of EphA4 antagonistic agents. However, with a K_{D} value of about 800-1000 nM, the linear KYL peptide lacks desired features, and as such, is not ideally suited as a platform for therapeutic development. In addition, both a phage display screen of a cyclic nonapeptide library and an NMR-based screen for smaller EphA4 peptidomimetic antagonists failed to yield peptides more potent than KYL.

In addition, to identifying EphA4 peptide antagonists of clinical relevancy, there is also a need to ensure these peptides are designed in a manner that prevents or reduces proteolysis in the circulating blood, prevents or reduces premature clearance through the kidneys, and/or prevents or reduces immunogenicity and the generation of neutralizing antibodies. Furthermore, issues regarding low bioavailability, including the peptide's inability to easily cross membrane barriers such as the intestinal and blood-brain barriers should also be addressed when developing EphA4 peptide antagonists useful for therapeutic applications. To date, no known EphA4 peptide antagonist appears to satisfy these physiological stability and bioavailability criteria to the extent that these peptides are recognized as clinically useful drugs.

Therefore, there is still a need to identify EphA4 peptide antagonists that possess the required potency and stability in biological systems to make them suitable therapeutic agents in the treatment of neurodegenerative disorders, neurotoxicity, nerve regeneration and cancer.

### SUMMARY

Some embodiments provide an EphA4 receptor antagonist comprising a chemical entity having the structure of Formula I, βAPYCVYZ¹βASWSCZ² (SEQ ID NO: 22), or a pharmaceutically acceptable salt thereof, wherein Z¹ is R; Z² is L¹-PEG or GGK(lauroyl-γEγE)G-NH₂ (SEQ ID NO: 23); and L¹ is a linker.

Other aspects disclose a pharmaceutical composition comprising one or more EphA4 receptor antagonists disclosed herein. A pharmaceutical composition disclosed can further comprises one or more pharmaceutical acceptable carriers.

Other aspects disclose a method of treating an EphA4-based disease, disorder or pathology. The disclosed method can comprise administering a EphA4 receptor antagonist disclosed herein or a pharmaceutical composition disclosed herein to an individual in need thereof. Administration of a EphA4 receptor antagonist disclosed herein or a pharmaceutical composition disclosed herein reduces one or more symptoms associated with the EphA4-based disease, disorder or pathology. An EphA4-based disease, disorder or pathology includes, without limitation, a neurodegenerative disease, a hearing loss, a promotion of nerve regeneration, a promotion of neuroprotection, or a cancer.

Other aspects disclose a method of treating a neurodegenerative disease. The disclosed method can comprise administering a EphA4 receptor antagonist disclosed herein or a pharmaceutical composition disclosed herein to an individual in need thereof. Administration of a EphA4 receptor antagonist disclosed herein or a pharmaceutical composition disclosed herein reduces one or more symptoms associated with the neurodegenerative disease.

Other aspects disclose a method of treating a hearing loss. The disclosed method can comprise administering a EphA4 receptor antagonist disclosed herein or a pharmaceutical composition disclosed herein to an individual in need thereof. Administration of a EphA4 receptor antagonist disclosed herein or a pharmaceutical composition disclosed herein reduces one or more symptoms associated with the hearing loss.

Other aspects disclose a method of promoting nerve regeneration. The disclosed method can comprise administering a EphA4 receptor antagonist disclosed herein or a pharmaceutical composition disclosed herein to an individual in need thereof. Administration of a EphA4 receptor antagonist disclosed herein or a pharmaceutical composition disclosed herein stimulates of facilitates neuronal differentiation and/or growth, thereby promoting nerve regeneration.

Other aspects disclose a method of promoting neuroprotection. The disclosed method can comprise administering a EphA4 receptor antagonist disclosed herein or a pharmaceutical composition disclosed herein to an individual in need thereof. Administration of a EphA4 receptor antagonist disclosed herein or a pharmaceutical composition disclosed herein protects neurons or nerve tissue from damage, thereby promoting neuroprotection.

Other aspects disclose a method of treating a cancer. The disclosed method can comprise administering a EphA4 receptor antagonist disclosed herein or a pharmaceutical composition disclosed herein to an individual in need thereof. Administration of a EphA4 receptor antagonist disclosed herein or a pharmaceutical composition disclosed herein reduces one or more symptoms associated with the cancer.

Other aspects disclose a use of a EphA4 receptor antagonist disclosed herein in the manufacture of a medicament for treating an EphA4-based disease, disorder or pathology. Other aspects disclose a use of a EphA4 receptor antagonist disclosed herein in the manufacture of a medicament for treating a neurodegenerative disease. Other aspects disclose a use of a EphA4 receptor antagonist disclosed herein in the manufacture of a medicament for treating a hearing loss. Other aspects disclose a use of a EphA4 receptor antagonist disclosed herein in the manufacture of a medicament for treating a cancer.

Other aspects disclose a use of a modified EphA4 receptor antagonist disclosed herein or a pharmaceutical composition disclosed herein in the treatment of an EphA4-based disease, disorder or pathology. Other aspects of the present specification disclose a use of a modified EphA4 receptor antagonist disclosed herein or a pharmaceutical composition disclosed herein in the treatment of a neurodegenerative disease. Other aspects of the present specification disclose a use of a modified EphA4 receptor antagonist disclosed herein or a pharmaceutical composition disclosed herein in the treatment of a hearing loss. Other aspects of the present specification disclose a use of a modified EphA4 receptor antagonist disclosed herein or a pharmaceutical composition disclosed herein in the treatment of a cancer.

Other aspects disclose a use of a EphA4 receptor antagonist disclosed herein in the manufacture of a medicament for promoting nerve regeneration. Other aspects disclose a use of a EphA4 receptor antagonist disclosed herein in the manufacture of a medicament for promoting neuroprotection.

Other aspects disclose a use of a EphA4 receptor antagonist disclosed herein or a pharmaceutical composition disclosed herein in the promotion of nerve regeneration. Other aspects disclose a use of a EphA4 receptor antagonist disclosed herein or a pharmaceutical composition disclosed herein in the promotion of neuroprotection.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1A shows a model of the structure of compound 2 in complex with the EphA4 ligand-binding domain shows that the side chain of Arg 7 and the C-terminal GGKG linker sequence (SEQ ID NO: 24) are exposed to the solvent and do not interact with EphA4. The GGKG sequence (SEQ ID NO: 24) also does not hinder the binding of the core of the APY-d2 peptide. Arrows point to Arg 7 and the C-terminus of the APY-d2 core. Modeling was done using the structure of APY-d3 in complex with the EphA4 ligand-binding domain (PDB 5JR2). Figure discloses “GGKG” as SEQ ID NO: 24.

FIGS. 1B-D show ELISAs indicating inhibition of ephrin-A5 AP binding to immobilized EphA4 Fc by different concentrations of compound 1, a derivative of APY-d2 with Lys replacing Arg 7 (B); compound 2, the derivative of APY-d2 with a C-terminal GGKG sequence (SEQ ID NO: 24) (C); and compound 3, a derivative of APY-d3 with Lys replacing Arg 7 (D). The curves for the derivative compounds are compared to the curves for the corresponding APY-d2 or APY-d3 obtained in parallel in the same ELISA experiments (which are a subset of the ELISA experiments used to calculate the overall IC₅₀ values shown in Table 1). The graphs show averages±SE of data from n independent experiments, each including triplicate measurements. Average IC₅₀ values±SE (nM) were calculated from multiparameter curve fitting of the combined data. ****, P<0.0001 for the comparison of the best fit IC₅₀ values with IC₅₀ value for APY-d2 (dark blue) or APY-d3 (gray) shown in the same panel, calculated using the extra sum-of-squares F test. The IC₅₀ value for compound 2 is not significantly different from that for APY-d2 in C.

FIGS. 2A-C shows ELISAs comparing the ability of the compounds to inhibit binding of ephrin-A5 fused to alkaline phosphatase (ephrin-A5-AP) to the immobilized EphA4 extracellular domain fused to the Fc portion of an antibody (EphA4-Fc). The graphs show averages±SE from n independent experiments, each including triplicate measurements. IC₅₀ values±SE (nM) were calculated from multiparameter curve fitting of the combined data. The curves for the albumin-binding compounds (orange) are compared to the curves for APY-d2 (dark blue) and/or APY-d3 (gray) compounds obtained in parallel in the same ELISA experiments (which are a subset of the ELISA experiments used to calculate the overall IC₅₀ values shown in Table 1).

FIGS. 3A-D show (A) Lipidated compounds administered intravenously. (B-C) Lipidated compounds or serum albumin-binding compound 21 administered intraperitoneally. (D) PEGylated compounds administered intraperitoneally. The data points show the percentage of injected compound remaining in the mouse blood at different times after administration. The concentration of the injected compound in a blood volume of 2.5 mL, which is the approximate blood volume of a mouse, was considered to be the concentration at the 0 time point and taken as 100% (empty circle). Approximate half-lives were calculated using a one phase decay equation. The graphs show averages±SE from 2-7 measurements (from n mice)±SE. The two rightmost panels in (D) show the data for APY-d3-PEG4 in a linear or logarithmic scale, with the rightmost panel also showing the compound concentrations in the mouse blood at different times after injection of 0.8 mg compound.

FIGS. 4A-E show (A) Lipidated compound 15. (B-E) PEGylated compounds. G, glycine; K, lysine; γE, γ-glutamic acid; βA, β-Alanine; Ahx, aminohexanoic acid; n indicates repeats of ethylene glycol. Figures disclose SEQ ID NOS 15, and 42-45, respectively, in order of appearance.

FIGS. 5A-C show (A) ELISAs comparing PEGylated APY-d3 with APY-d3 for their ability to inhibit binding of ephrinA5-AP to EphA4-Fc. The graphs show averages±SE from n independent experiments, each including triplicate measurements. IC₅₀ values±SE (nM) were calculated from multiparameter curve fitting of the combined data. The curves for the PEGylated compounds (red) are compared to the curves for APY-d3 (gray) obtained in parallel in the same ELISA experiments (which are a subset of the ELISA experiments used to calculate the overall IC₅₀ values shown in Table 1). (B) Ephrin-A5 AP binding to EphA receptors and ephrin-B2 AP binding to EphB receptors in the presence of 1.9 μM APY-d3-PEG4 (a concentration ˜100 fold higher than the IC₅₀ value in (A)). Values are normalized to ephrin binding without compound. The graph shows averages and SEs from 3-6 measurements (with each measurement shown as a dot). (C) ELISA measuring EphA4 activation in cells. Dose-response curves obtained from quantifications of EphA4 tyrosine phosphorylation induced by ephrin-A5 Fc in immortalized cells stably expressing FLAG-tagged EphA4 (HEK-EphA4 cells). The cells were treated with the indicated concentrations of APY-d3-PEG4 or APY-d3 for 20 min and then with 0.5 mg/μL ephrin-A5 Fc for 10 min, or with Fc as a control; “0” indicates no compound treatment. EphA4 was captured from the cell lysates in wells coated with an anti-FLAG antibody and EphA4 phosphorylation on tyrosine 596 (pY596) was measured with an antibody to the conserved pY588 motif of EphA2, followed by an HRP-conjugated secondary antibody. The graph shows averages±SE from n independent experiments. IC₅₀ values±SE (nM) were calculated from multiparameter curve fitting of the combined data. The APY-d3-PEG4 is ˜2.5 fold less potent than APY-d3; * P=0.42.

FIGS. 6A-D show ELISAs comparing the ability of the compounds to inhibit binding of ephrin-A5 AP to EphA4 Fc in the presence of 40 mg/mL delipidated HSA (A), 6.5 mM methyl-b-cyclodextrin (B), or 20 mg/mL BSA (C) in comparison to buffer. The graphs show averages±SE from n independent experiments, each including triplicate measurements. IC₅₀ values±SE (nM) were calculated from multiparameter curve fitting of the combined data. The curves in the presence of human serum albumin (light blue), methyl-β-cyclodextrin (pink), and BSA (purple) are compared to the curves in buffer (black) obtained in parallel in the same ELISA experiments (which are a subset of the ELISA experiments used to calculate the overall IC₅₀ values shown in Table 1). *, P<0.05; **, P<0.01; ***, P<0.001; ****, P<0.0001.

FIG. 7 shows After intraperitoneal injection of compound 14 in a mouse, blood was taken at 1, 2 and 4 hours. Plasma was used in ELISAs to determine the apparent IC₅₀ value for inhibition of ephrin-A5 AP binding to immobilized EphA4 Fc. The 0 time point, taken as 100%, was obtained by measuring the IC₅₀ value for the compound diluted in plasma in vitro, estimating that all of the injected compound reaches the blood and a blood volume of 2.5 mL, which is the approximate blood volume of a mouse.

### DETAILED DESCRIPTION

EphA4 is a particularly promiscuous receptor that can bind both ephrin-A and ephrin-B ligands. The difficulties in obtaining submicromolar EphA4 antagonists are likely due to the nature of the ephrin-binding pocket of EphA4 to accommodate the binding of multiple ligands. Previously prepared small cyclic peptides showed good resistance to proteolytic degradation and high potency. However, once in the blood circulation, cyclic peptides such as APY-d3 are typically excreted through the kidneys within a few minutes. Thus, improvements are needed to increase the in vivo half-life of EphA4 antagonists. Described herein are EphA4 antagonists with dramatically improved in vivo half-life in comparison to APY-d3 while maintaining high potency.

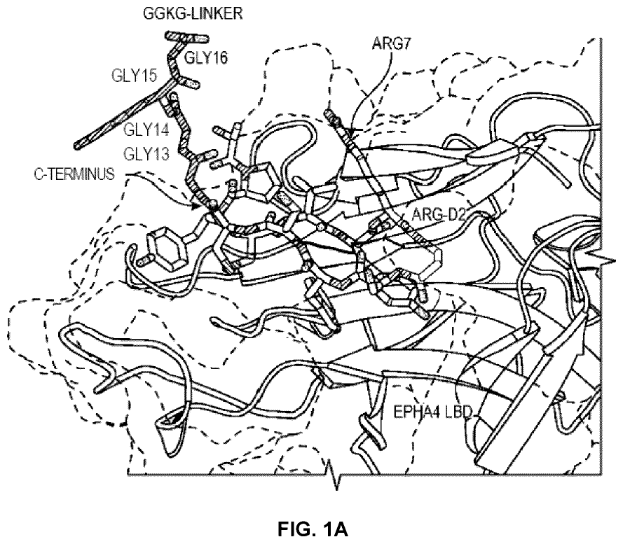

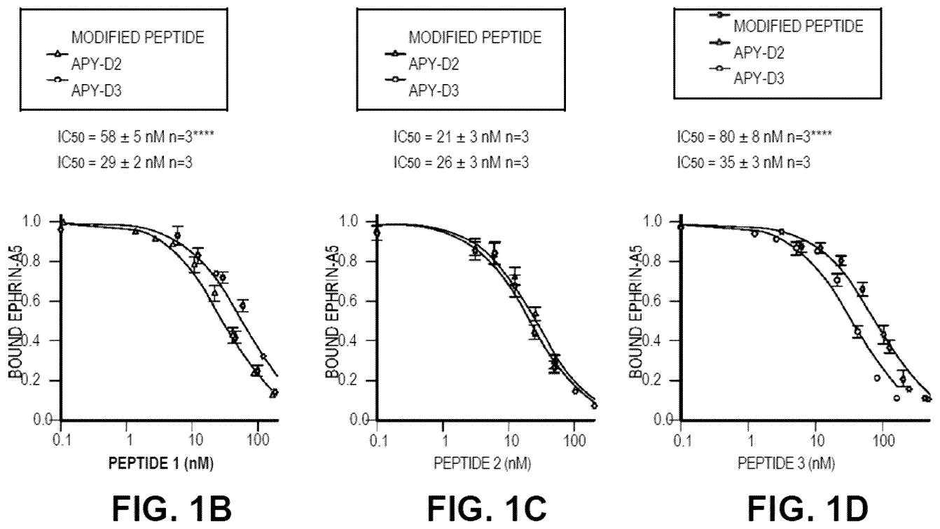

EphA4 antagonists with Arg7 in APY-d2 and APY-d3 replaced with Lys (compounds 1 and 3 in Table 1) were prepared to evaluate including the side chain amino group in the Lys which could be used for modification. It was demonstrated that this modification decreased potency (IC₅₀) by ˜2 folds when comparing in the same experiments compound 1 to APY-d2 and compound 3 to APY-d3 in ELISA experiments measuring inhibition of ephrin-A5 AP binding to immobilized EphA4 Fc (FIGS. 1B and D). In addition, a Lys was appended to the C terminus of APY-d2 via a GlyGly spacer (compound 2 in Table 1). It was demonstrated that compound 2 inhibits binding of the ephrin-A5 ligand to EphA4 with a potency that is similar to that of APY-d2 tested in parallel (FIG. 1C).

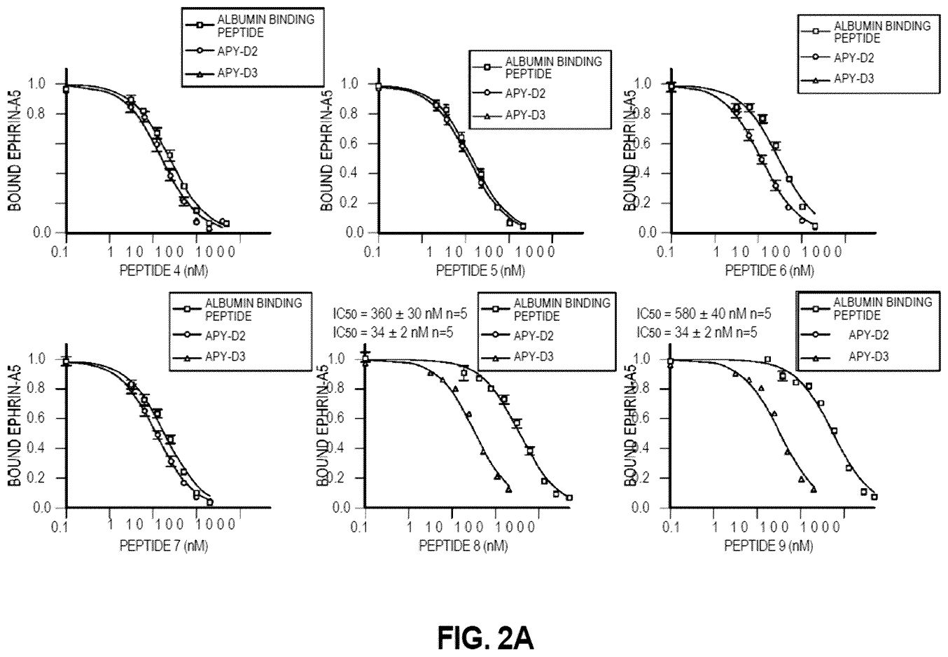

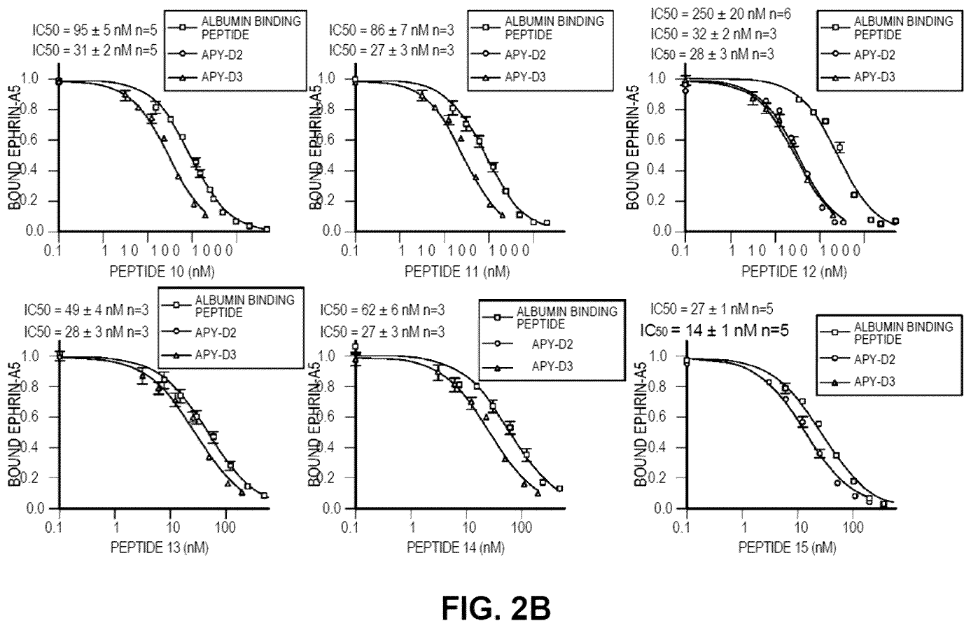

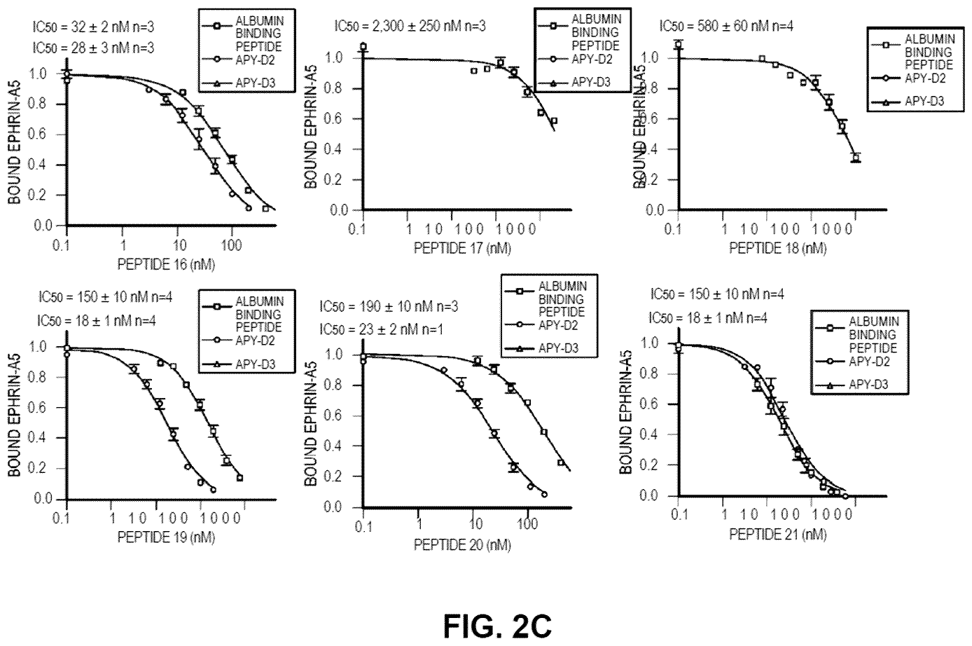

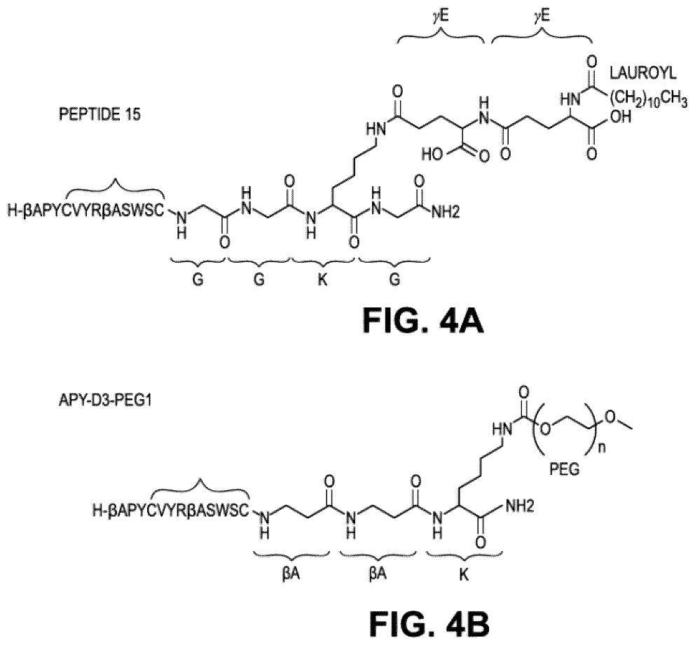

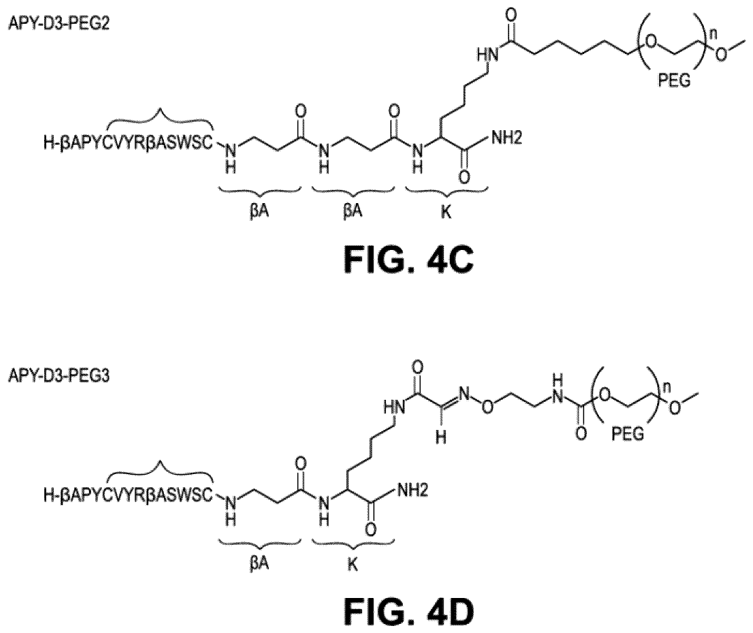

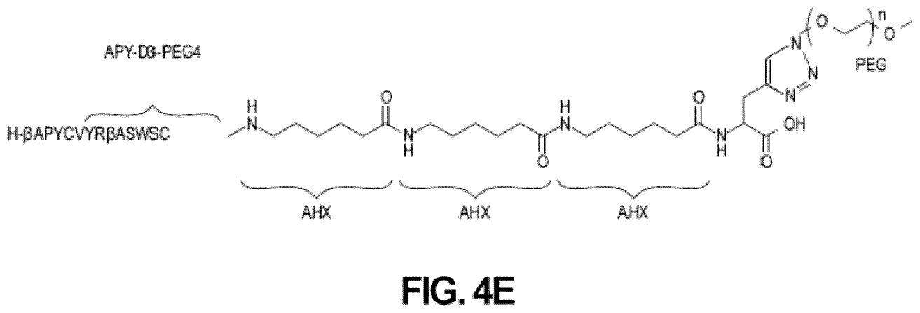

In view of the preceding information, attachment of a C8 to C16 lipid chain to the side chain amino group of Lys was implemented to determine whether such modification could increase in vivo half-life in comparison to APY-d3. A series of lipidated compounds were prepared and inhibition of EphA4-ephrin-A5 interaction was measured by ELISA (FIGS. 2A-C and Table 1). Attachment of the C8 lipid octanoic acid to the side chain of Lys7 alleviated the loss in potency due to the Lys to Arg substitution (compare compound 4 with compound 3). Furthermore, the C-terminal GGRG sequence (SEQ ID NO: 46) added to compound 4 to yield compound 5 can enhance solubility without detrimental effects on potency. Replacement of the octanoic acid in compound 4 with octanyl-glycine in compound 6 did not substantially affect potency. APY-d3 was also modified with octanoic acid attached to the lysine side chain of an added C-terminal GGKG sequence (SEQ ID NO: 24) in compound 7, which has similar potency as the other compounds containing a C8 lipid. The C12 lipid lauric acid, or lauryl-glycine, attached to Lys7 in compounds 8 and 9, respectively, dramatically decreased potency by >10-fold compared to APY-d3 similarly modified with a C8 lipid. Introduction of a spacer such as β-aspartic acid (βAsp) or γ-glutamic acid (γGlu) improved potency by several fold (compare compounds 10 and 11 with compound 8). Addition of lauric acid to the APY-d3 C-terminus in compound 12 also resulted in substantial loss in potency compared to the corresponding octanoyl compound 7. However, the βAsp and γGlu spacers in compounds 13 and 14, respectively, greatly improved the potency of the compounds with C-terminal lauric acid. Finally, a longer spacer consisting of two γGlu residues (FIG. 4A) resulted in better potency than a single γGlu (comparing compound 14 with compound 15, which has a potency comparable to APY-d3). An increase in the size of the C12 lipid in compound 15 to the C14 lipid myristoyl acid attached through a γGluγGlu spacer in compound 16 further decreased potency by 2-3-fold. Finally, the C16 lipid palmitic acid proved to be very detrimental to compound potency even with a γGluγGlu spacer, particularly when attached to Lys7 (compounds 17 and 18). As observed for the corresponding lauroyl compounds, a longer spacer consisting of two γGlu residues resulted in a ˜3 fold better potency compared to a single γGlu (compound 19 versus compound 18), although compound 19 remains 5-6-fold less potent than compound 15. A further increase in the length of the spacer by including an additional Gly-Ser in the C-terminal linker of compound 19 to obtain compound 20 did not further improve potency.

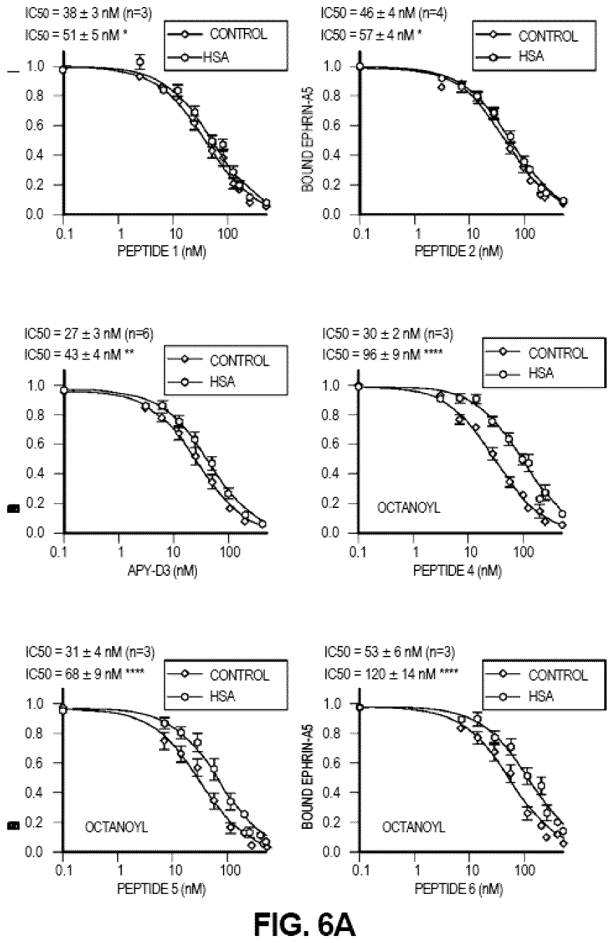

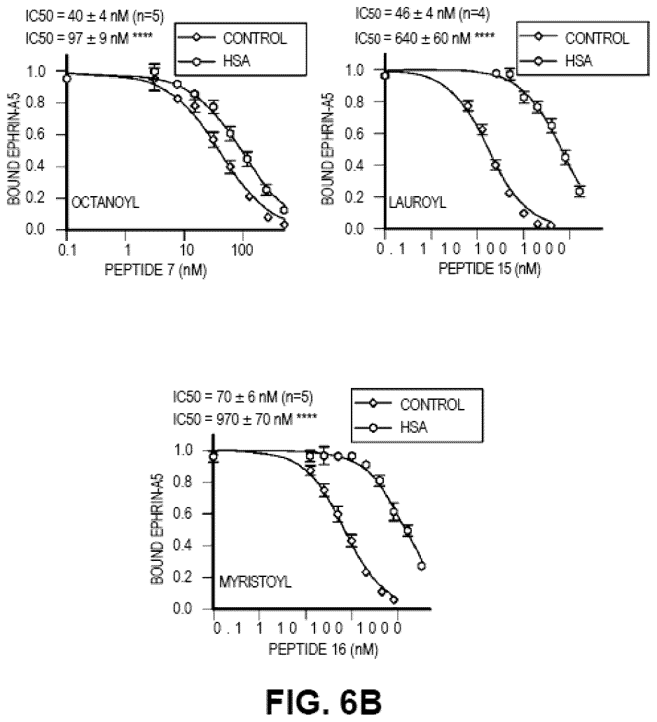

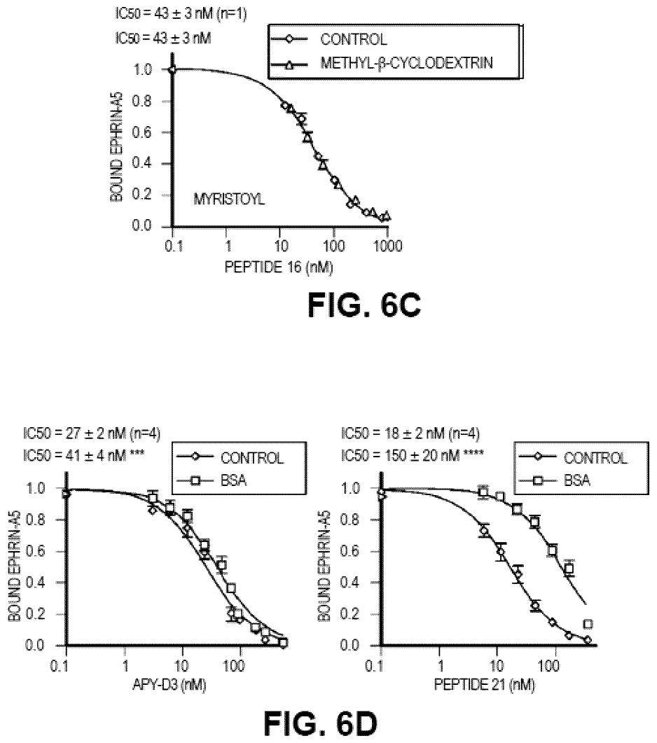

The effects of delipidated human serum albumin (HSA) on the inhibitory potency of some of the compounds was commenced to determine whether albumin binding to the lipidated compounds affects their ability to bind EphA4. By comparing compounds tested in parallel in the presence and in the absence of 40 mg/mL HSA (which is the concentration present in the blood), it was determined that HSA has only a very small effect on the potency of the non-lipidated compounds 1, 2 and APY-d3 examined as controls (FIG. 6A and Table 1). HSA decreased the potency of the octanoyl compounds 4, 5, 6 and 7 by 2-3 fold, suggesting that the binding of serum albumin to these compounds interferes with EphA4 binding (FIGS. 6A-B and Table 1). In contrast, HSA dramatically decreased the potency of the lauroyl compound 15 and the myristoyl compound 16. The effects of HSA on the palmitoylated compounds were not determined, given their low potency and poor solubility. Overall, the data suggests that C-terminal lipidation can be better tolerated than lipidation on Lys7, that a shorter lipid is better tolerated than a longer lipid, and that βAsp or γGlu spacers can alleviate the decrease in potency caused by longer lipids.

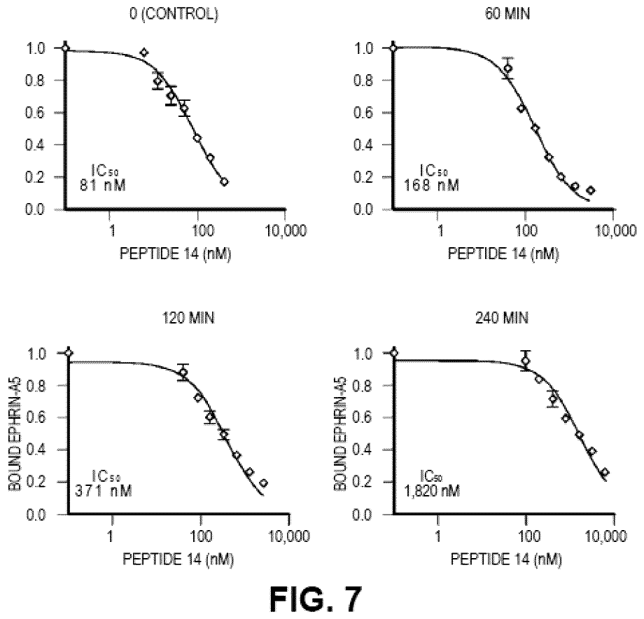

An approach was devised to determine the effects of the various lipid modifications on compound persistence in the blood circulation by evaluating the concentration of active compound in mouse blood by taking advantage of the ELISA measuring inhibition of EphA4-ephrinA5 interaction. The approach involves deducing the concentration of compound remaining in the blood at various time points after administration by comparing the apparent IC₅₀ value for the compound remaining in plasma with the IC₅₀ value of a control obtained using mouse plasma spiked in vitro with a known compound concentration (corresponding to the theoretical concentration at the 0 time point, based on the amount of injected compound and a mouse blood volume of 2.5 mL) (FIG. 7). For example, the 5-fold increase in the apparent IC₅₀ value using plasma obtained 120 min after compound 14 administration indicates a 5-fold decrease in compound concentration in the blood, and therefore that 20% of the injected compound remains in the blood after 2 hours (Table 3).

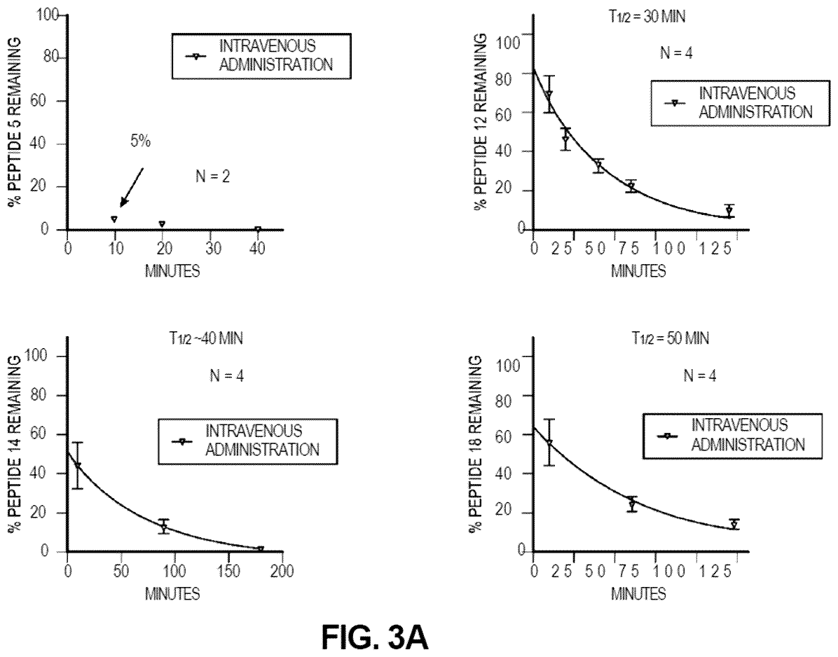

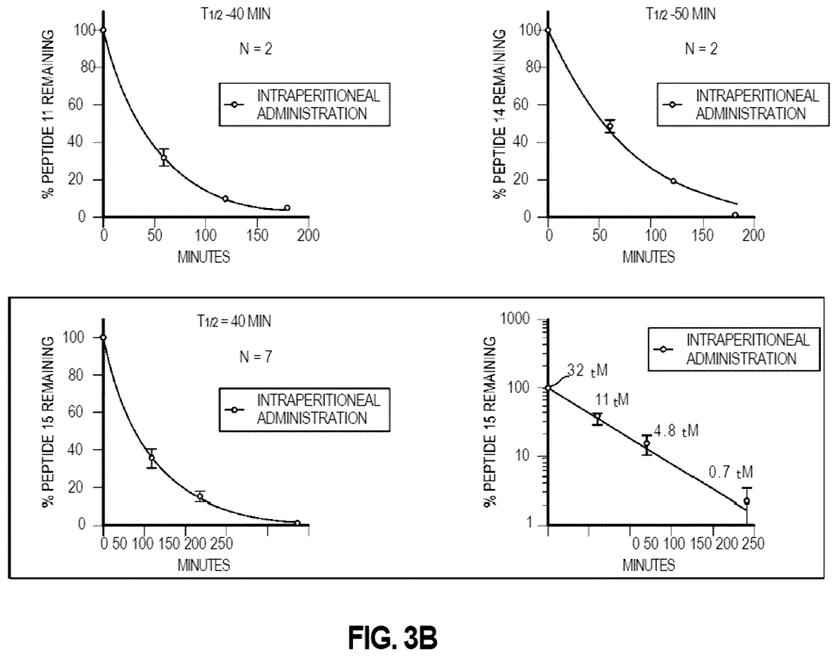

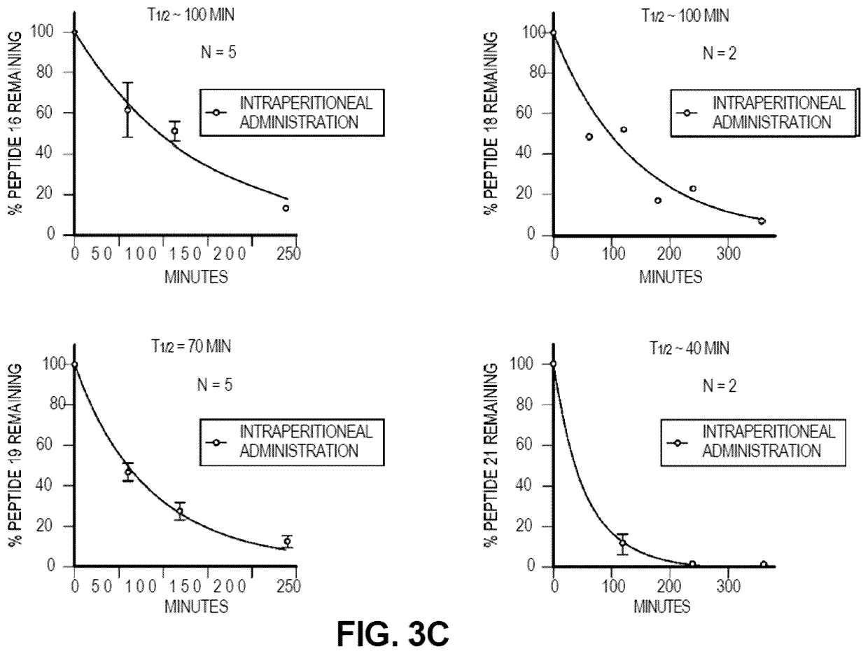

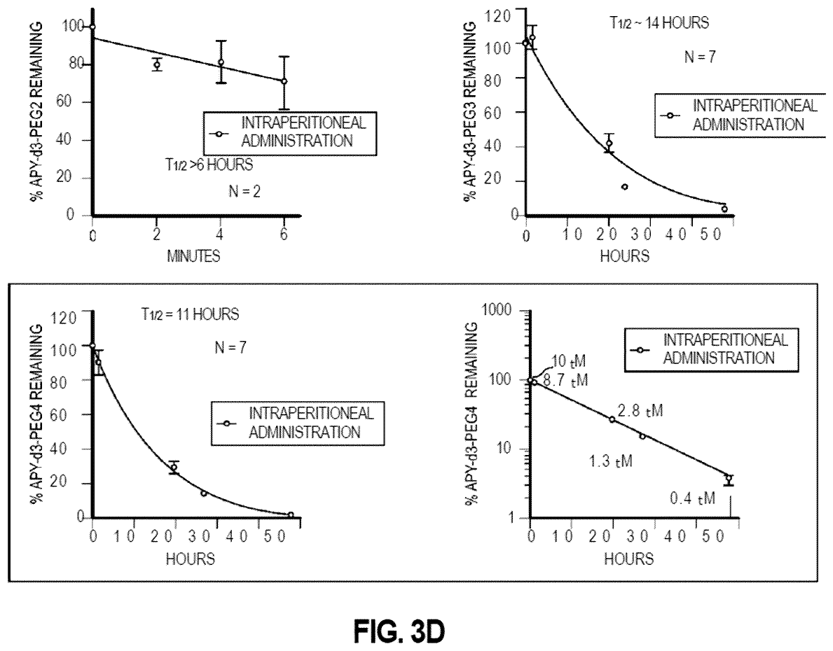

The in vivo half-life of representative lipidated compounds (FIGS. 3A-C and Table 1) was examined. For compound 5 modified on Lys7 with octanoic acid (which is the shortest lipid used), only 5% of compound 5 remained in the blood 10 min after intravenous injection of compound 5 dissolved in PBS supplemented with 5% DMSO. However, the solubility of compounds modified with longer lipids is poor in PBS containing 5% DMSO at the concentrations needed for administration. Therefore, a formulation including 20-40 mg/mL bovine serum albumin was used, which was sufficient to eliminate visible insoluble particles. For compounds modified at the C-terminus with lauric acid, half-lives of 0.5-0.7 hours were measured for compounds 12 and 14 injected intravenously and 0.5-1 hours for compounds 11, 14 and 15 injected intraperitoneally. C-terminal attachment of the C14 lipid myristoyl acid through a γGluγGlu spacer in compound 16 prolongs to ˜2 hours the in vivo half-life of the compounds after intraperitoneal injection. Formulation of compound 16 with methyl-β-cyclodextrin, a biocompatible cyclic oligosaccharide used to improve the solubility and bioavailability of hydrophobic drugs (6), yielded similar in vivo half-life as the formulation with serum albumin. Of note, methyl-b-cyclodextrin does not affect the inhibitory potency of compound 16 (FIG. 6C). Finally, compounds 18 and 19 with C-terminal palmitic acid had a half-life of ˜0.8 hours after intravenous injection and 1 to 1.5 hours after intraperitoneal injection. The lauroyl compound 15 and the myristoyl compound 16 are the most promising of the lipidated compounds considering potency and in vivo half-life. These compounds, with IC₅₀ values of ˜50 and ˜80 nM, respectively, and a half-life in the circulation of 1 to 2 hours, could be injected intraperitoneally once or twice a day in mice to maintain a therapeutically effective blood concentration (0.5-1 μM) or less frequently to achieve high but transient levels in the blood, which could be desirable in some cases.

The C-terminus of APY-d3 was fused with the N-terminus of the albumin-binding cyclic peptide SA21 (RLIEDICLPRWGCLWEDD, where the two underlined Cys residues form a disulfide bond (SEQ ID NO: 25)) to promote complexation with serum albumin. The SA21 peptide has been reported to bind albumin with higher affinity than lipids (K_{d} 470 nM for human serum albumin. The resulting compound 21 appears to be as potent as APY-d3 (FIGS. 2A-C and Table 1). However, the potency was greatly (˜8-fold) decreased in the presence of 20 mg/mL bovine serum albumin (FIG. 6C), suggesting that serum albumin binding to the SA21 moiety strongly interferes with the binding of the APY-d3 moiety to EphA4. In addition, the in vivo half-life of compound 21 (˜40 min) is shorter than that of the best lipidated compounds (FIGS. 3B-C). This could be due to the presence of an arginine at the N-terminus of SA21, which can be susceptible to proteolytic cleavage separating the APY-d3 and SA21 moieties of compound 21, leading to the rapid excretion of the released APY-d3. These drawbacks have to be overcome in order to make compound 21 suitable for in vivo use.

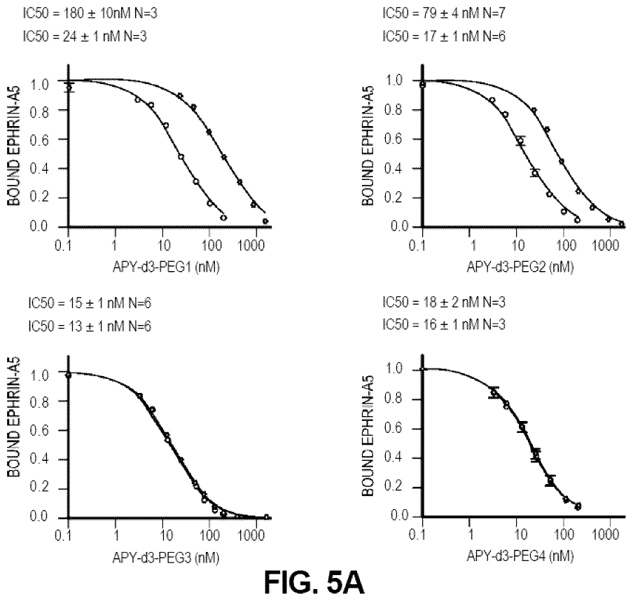

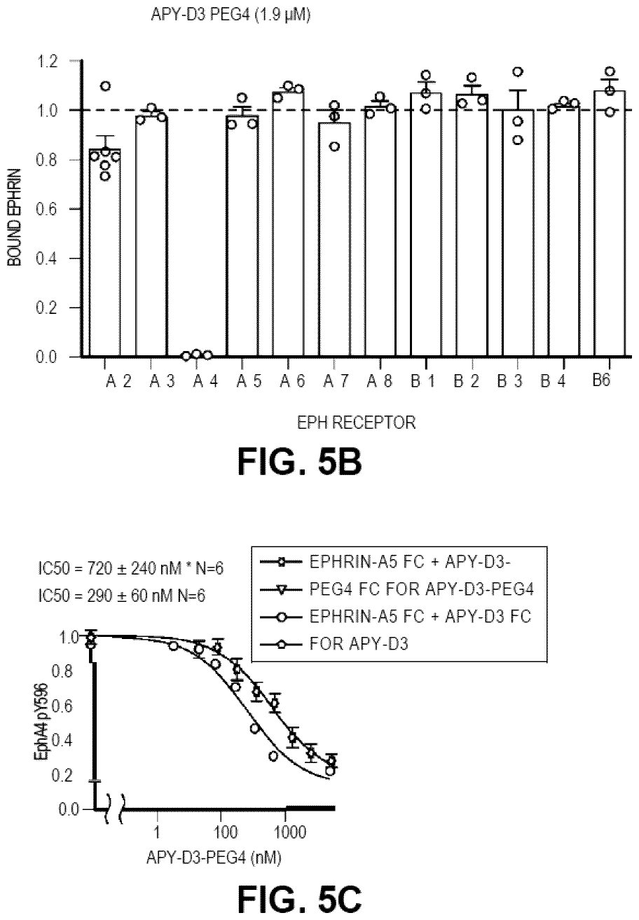

Extension of half-life using polyethylene glycol (PEG) was examined by conjugating a 30 kDa PEG to the C-terminus of APY-d3 using an amide formation reaction and two alkane linkers of different reactivity to afford APY-d3-PEG1 and APY-d3-PEG2 (FIGS. 4B and C). The PEG conjugate with the longer linker (APY-d3-PEG2) is the more potent of the two (FIG. 5A and Table 1), but with an IC₅₀ of ˜80 nM it is several fold less potent than non-PEGylated APY-d3. However, the in vivo half-life of APY-d3-PEG2 is longer than 6 hours (FIG. 3D).

The initial approach used to generate APY-d3-PEG1 and APY-d3-PEG2, which involves amide formation requires deprotection of the APY-d3 N-terminus after PEG conjugation. Two alternative strategies for PEG conjugation were developed, both of which can be carried out with a deprotected N-terminus thereby facilitating the synthesis of large amounts of PEGylated compounds. The first strategy involves conjugating the PEG to APY-d3 through a stable oxime linkage (FIG. 4D; APY-d3-PEG3). The aldehyde and aminooxy bioconjugation groups react quantitatively and because the PEG conjugation is the final step in the synthesis, a conjugate of acceptable purity can be produced. In addition, with this strategy the majority of the synthetic steps occur on the solid phase. This eliminates the need for repeated intermediate HPLC purifications, resulting in increased yields. The second strategy involves conjugating APY-d3 to PEG through a linker containing 3 amino-hexanoic acid groups and propargyl-glycine using click chemistry (FIG. 4E; APY-d3-PEG4). This conjugation can be carried out in solution, also with an unprotected N-terminus.

Unexpectedly, both APY-d3-PEG3 and APY-d3-PEG4 have similar potency as the non-PEGylated APY-d3 in ELISAs measuring inhibition of EphA4 ephrin-A5 interaction (FIG. 5A and Table 1). This is an improvement over the previously used PEGylation approach, which resulted in a several fold loss in potency. Most importantly, the in vivo half-lives of active APY-d3-PEG3 and APY-d3-PEG4 after intraperitoneal injection are ≥11 hours (FIG. 3C). Thus, APY-d3-PEG3 and APY-d3-PEG4 have superior characteristics compared to the best lipidated compounds when prolonged EphA4 inhibition is desired. Given the variability in observed potency of some batches of APY-d3-PEG3, APY-d3-PEG4 was selected for experiments to measure inhibition of ligand-induced EphA4 activation in cells

It was discovered that APY-d3-PEG4, even at high concentrations, selectively inhibits only EphA4 among the Eph receptors, as is the case for APY-d3 (FIG. 5B). Also, it was discovered that APY-d3-PEG4 inhibits EphA4 tyrosine phosphorylation (indicative of activation) in cells stimulated with ephrin-A5. The inhibitory potency of APY-d3-PEG4 is 2-3 fold lower than that of APY-d3 (IC₅₀ values of ˜700 nM for APY-d3-PEG4 and ˜300 nM for APY-d3; FIG. 5C). Nevertheless, intraperitoneal administration of ˜0.8 mg/mouse APY-d3-PEG4 results in an early blood concentration of ˜9 μM, which decreases to ˜3 μM after 20 hours and to ˜0.4 μM after 48 hours. Since 0.5 to 1 μM APY-d3-PEG4 is sufficient to substantially inhibit EphA4 tyrosine phosphorylation induced by ephrinA5-Fc stimulation in cells, these data suggest that APY-d3-PEG4 can be administered intraperitoneally every other day in order to maintain a concentration sufficient to inhibit EphA4 in vivo.

In some embodiments, compound 15 and APY-d3-are useful for studies of EphA4 inhibition in animal disease models.

Aspects of the present specification disclose, in part, EphA4 antagonists, that selectively bind to the ephrin-binding pocket in the EphA4 ligand binding domain. Selective binding includes binding properties such as, e.g., binding affinity and binding specificity.

In another embodiment, the binding affinity of compounds disclosed herein, can have an equilibrium disassociation constant for an EphA4 receptor of less than 500 nM. In an aspect of this embodiment, the binding affinity of compound disclosed herein, can have an equilibrium disassociation constant for an EphA4 receptor of, e.g., less than 500 nM, less than 450 nM, less than 400 nM, less than 350 nM, less than 300 nM, less than 250 nM, less than 200 nM, less than 150 nM, less than 100 nM, less than 75 nM, less than 50 nM, less than 25 nM, less than 20 nM, less than 15 nM, less than 10 nM, less than 5 nM, less than 1 nM, less than 0.5 nM, or less than 0.1 nM. In an aspect of this embodiment, the binding affinity of EphA4 antagonist, like a compound disclosed herein, can have an equilibrium disassociation constant for an EphA4 receptor of, e.g., about 0.1 nM to about 10 nM, about 0.1 nM to about 25 nM, about 0.1 nM to about 75 nM, about 0.1 nM to about 100 nM, about 0.1 nM to about 125 nM, about 0.1 nM to about 150 nM, about 0.5 nM to about 10 nM, about 0.5 nM to about 25 nM, about 0.5 nM to about 75 nM, about 0.5 nM to about 100 nM, about 0.5 nM to about 125 nM, about 0.5 nM to about 150 nM, about 1 nM to about 10 nM, about 1 nM to about 25 nM, about 1 nM to about 75 nM, about 1 nM to about 100 nM, about 1 nM to about 125 nM, about 1 nM to about 150 nM, about 5 nM to about 10 nM, about 5 nM to about 25 nM, about 5 nM to about 75 nM, about 5 nM to about 100 nM, about 5 nM to about 125 nM, about 5 nM to about 150 nM, about 10 nM to about 25 nM, about 10 nM to about 50 nM, about 10 nM to about 75 nM, about 10 nM to about 100 nM, about 10 nM to about 125 nM, about 10 nM to about 150 nM, about 10 nM to about 175 nM or about 10 nM to about 200 nM.

Aspects of the present specification disclose, in part, lipidation. Lipidation involves the formation of a stable covalent attachment of one or more lipid moieties to a compound of interest.

The presence of one or more lipid moieties can alter various physicochemical properties of a compound including, without limitation, increasing the size and molecular weight of the compound. Such modifications can change the pharmacokinetic and pharmacodynamic properties of the compound conjugate while retaining its functionality as well as improve its metabolic stability, decrease its clearance rate, decrease its susceptibility to proteolysis, alter its self-assembling propensities, improve its membrane permeability, reduce its immunogenicity and antigenicity and/or improve its bioavailability relative to the non-lipidated compound. The masking of potential antigenic sites by one or more lipid moieties also can decrease the generation of neutralizing antibodies against the compound.

Formation of a lipidated-conjugated compound involves the acylation of a functional group present on a side chain of the compound with a long-chain lipid. Covalent attachment of an activated a lipid is generally made at the œ or & amino groups of lysine, a β-aspartic acid, a γ-glutamic acid, or any other amino acid have a free amino group; the N-terminal amino group of cysteine, histidine, arginine, aspartic acid, glutamic acid, serine, threonine, tyrosine; or the C-terminal carboxylic acid. Reagents and methods of attaching a lipid are described in Zhang and Bulaj, Converting Peptides into Drug Leads by Lipidation, Curr. Med. Chem. 19:1602-1618 (2012), which is hereby incorporated by reference in its entirety.

Typically, the lipid moiety used is a fatty acid, a lipid comprises a carboxylic acid with a long unbranched hydrocarbon chain, which can be either saturated or unsaturated. Examples of fatty acids include, without limitation, hexanoic (caproic) acid (6:0), heptanoic (enanthic) acid (7:0), octanoic (capryllic) acid (8:0), nonanoic (pelargonic) acid (9:0), decanoic (capric) acid (10:0), undecanoic (undecylic) acid (11:0), dodecanoic (lauric) acid (12:0), tridecanoic (tridecylic) acid (13:0), tetradecanoic (myristic) acid (14:0), myristoleic acid (14:1), pentadecanoic (pentadecyclic) acid (15:0), hexadecanoic (palmitic) acid (16:0), palmitoleic acid (16:1), sapienic acid (16:1), heptadecanoic (margaric) acid (17:0), octadecanoic (stearic) acid (18:0), oleic acid (18:1), elaidic acid (18:1), vaccenic acid (18:1), linoleic acid (18:2), linoelaidic acid (18:2), α-linolenic acid (18:3), γ-linolenic acid (18:3), stearidonic acid (18:4), nonadecylic acid (19:0), arachidic acid (20:0), eicosenoic acid (20:1), dihomo-γ-linolenic acid (20:3), mead acid (20:3), arachidonic acid (20:4), eicosapentaenoic acid (20:5), heneicosylic acid (21:0), behenic acid (22:0), erucic acid (22:1), docosahexaenoic acid (22:6), tricosylic acid (23:0), lignoceric acid (24:0), nervonic acid (24:1), pentacosylic acid (25:0), cerotic acid (26:0), heptacosylic acid (27:0), montanic acid (28:0), nonacosylic acid (29:0), melissic acid (30:0), henatriacontylic acid (31:0), lacceroic acid (32:0), psyllic acid (33:0), geddic acid (34:0), ceroplastic acid (35:0), and hexatriacontylic acid (36:0).

In an embodiment, a lipid can be a pharmaceutically-acceptable saturated or unsaturated fatty acid or diacid. In aspects of this embodiment, a saturated or unsaturated fatty acid or diacid comprises, e.g., at least 6, at least 7, at least 8, at least 9, at least 10, at least 11, at least 12, at least 13, at least 14, at least 15, at least 16, at least 17, at least 18, at least 19, at least 20, at least 22, at least 24, at least 26, at least 28, or at least 30 carbon atoms. In other aspects of this embodiment, a saturated or unsaturated fatty acid comprises, e.g., between 4 and 24 carbon atoms, between 5 and 24 carbon atoms, between 6 and 24 carbon atoms, between 7 and 24 carbon atoms, between 8 and 24 carbon atoms, between 9 and 24 carbon atoms, between 10 and 24 carbon atoms, between 11 and 24 carbon atoms, between 12 and 24 carbon atoms, between 13 and 24 carbon atoms, between 14 and 24 carbon atoms, between 15 and 24 carbon atoms, between 16 and 24 carbon atoms, between 4 and 22 carbon atoms, between 5 and 22 carbon atoms, between 6 and 22 carbon atoms, between 7 and 22 carbon atoms, between 8 and 22 carbon atoms, between 9 and 22 carbon atoms, between 10 and 22 carbon atoms, between 11 and 22 carbon atoms, between 12 and 22 carbon atoms, between 13 and 22 carbon atoms, between 14 and 22 carbon atoms, between 15 and 22 carbon atoms, between 16 and 22 carbon atoms, between 4 and 20 carbon atoms, between 5 and 20 carbon atoms, between 6 and 20 carbon atoms, between 7 and 20 carbon atoms, between 8 and 20 carbon atoms, between 9 and 20 carbon atoms, between 10 and 20 carbon atoms, between 11 and 20 carbon atoms, between 12 and 20 carbon atoms, between 13 and 20 carbon atoms, between 14 and 20 carbon atoms, between 15 and 20 carbon atoms, between 16 and 20 carbon atoms, between 4 and 18 carbon atoms, between 5 and 18 carbon atoms, between 6 and 18 carbon atoms, between 7 and 18 carbon atoms, between 8 and 18 carbon atoms, between 9 and 18 carbon atoms, between 10 and 18 carbon atoms, between 11 and 18 carbon atoms, between 12 and 18 carbon atoms, between 13 and 18 carbon atoms, between 14 and 18 carbon atoms, between 15 and 18 carbon atoms, between 16 and 18 carbon atoms, between 4 and 16 carbon atoms, between 5 and 16 carbon atoms, between 6 and 16 carbon atoms, between 7 and 16 carbon atoms, between 8 and 16 carbon atoms, between 9 and 16 carbon atoms, between 10 and 16 carbon atoms, between 11 and 16 carbon atoms, between 12 and 16 carbon atoms, between 13 and 16 carbon atoms, between 14 and 16 carbon atoms, between 4 and 14 carbon atoms, between 5 and 14 carbon atoms, between 6 and 14 carbon atoms, between 7 and 14 carbon atoms, between 8 and 14 carbon atoms, between 9 and 14 carbon atoms, between 10 and 14 carbon atoms, between 11 and 14 carbon atoms, between 12 and 14 carbon atoms, between 4 and 12 carbon atoms, between 5 and 12 carbon atoms, between 6 and 12 carbon atoms, between 7 and 12 carbon atoms, between 8 and 12 carbon atoms, between 9 and 12 carbon atoms, or between 10 and 12 carbon atoms. If unsaturated, the fatty acid can have, e.g., 1 or more, 2 or more, 3 or more, 4 or more, 5 or more, or 6 or more double bonds.

Aspects of the present specification disclose, in part, PEGylation. PEGylation involves the formation of a stable covalent attachment of one or more synthetic poly(ethylene glycol) (PEG) polymers to a compound of interest. PEG polymers are typically biologically inert, non-immunogenic molecules that confer greater water solubility to compounds by forming a hydrophilic shell. PEG polymers are synthesized by the polymerization of ethylene oxide and are commercially available over a wide range of molecular weights from 100 g/mol to 10,000,000 g/mol. PEG polymers can have a linear or branched structure. Branched PEG polymers can increase the size of the total conjugate without resultant increase in number of attachment sites and have been shown to improve stability in response to changes in pH, proteolytic digestion, and temperature change as compared to linear PEG polymers. Depending on how one chooses to define the constituent monomer (as ethylene glycol, ethylene oxide or oxyethylene), PEG polymers are also known as polyethylene oxide (PEO) polymers and polyoxyethylene (POE) polymers. In some embodiments, the PEG polymer includes a terminal OCH₃ instead of a terminal OH. In some embodiments, the PEG polymer includes a terminal OCH₂CH₃ instead of a terminal OH. In some embodiments, the PEG polymer is a linear PEG polymer. In some embodiments, the EphA4 receptor antagonist comprises a branched PEG polymer using Y-PEG40K-MAL (Product #JKA0002, Sigma-Aldrich) in the preparation of the EphA4 receptor antagonist.

The presence of one or more PEG polymers can alter various physicochemical properties of a compound including, without limitation, increasing the size and molecular weight of the compound.

Formation of a PEG-conjugated compound involves the activation of PEG polymers by preparing derivatives with functional groups at one or both of the terminal ends of the polymers. PEG polymer derivatives with functional groups at both terminal ends can be homobifunctional (identical reactive groups at either end) and heterobifunctional (different reactive groups at either end). PEG polymers can be an oxidized, reduced, aminated and/or hydrazide derivative. Useful functional groups include, without limitation, amine reactive PEG polymers activate by, e.g., the presence of an N-hydroxy-succinimide (NHS) ester that react with amine groups; and sulfhydryl-reactive PEG polymers activate by, e.g., the presence of a maleimide group that react sulfhydryl groups. Covalent attachment of an activated PEG polymer is generally made at the co or & amino groups of lysine, a β-aspartic acid, a γ-glutamic acid, or any other amino acid have a free amino group; the N-terminal amino group of cysteine, histidine, arginine, aspartic acid, glutamic acid, serine, threonine, tyrosine; or the C-terminal carboxylic acid. Among these amino acids, a choice for PEGylation is lysine and N-terminal amino group.

The exact molecular weight of a PEG polymer used to conjugate to a compound can vary. In aspects of this embodiment, the number of monomers comprising an activated PEG polymers useful to modify a compound disclosed herein can be, e.g., about 4 monomers, about 5 monomers, about 6 monomers, about 7 monomers, about 8 monomers, about 9 monomers, about 10 monomers, about 11 monomers, about 12 monomers, about 13 monomers, about 14 monomers, about 15 monomers, about 16 monomers, about 17 monomers, about 18 monomers, about 19 monomers, about 20 monomers, about 21 monomers, about 22 monomers, about 23 monomers, about 24 monomers, about 25 monomers, about 26 monomers, about 27 monomers, about 28 monomers, about 29 monomers or about 30 monomers. In other aspects of this embodiment, the number of monomers comprising an activated PEG polymers useful to modify a compound disclosed herein can be, e.g., at least 4 monomers, at least 5 monomers, at least 6 monomers, at least 7 monomers, at least 8 monomers, at least 9 monomers, at least 10 monomers, at least 11 monomers, at least 12 monomers, at least 13 monomers, at least 14 monomers, at least 15 monomers, at least 16 monomers, at least 17 monomers, at least 18 monomers, at least 19 monomers, at least 20 monomers, at least 21 monomers, at least 22 monomers, at least 23 monomers, at least 24 monomers, at least at least 25 monomers, at least 26 monomers, at least 27 monomers, at least 28 monomers, at least 29 monomers or at least 30 monomers. In yet other aspects of this embodiment, the number of monomers comprising an activated PEG polymers useful to modify a compound disclosed herein can be, e.g., at most 4 monomers, at most 5 monomers, at most 6 monomers, at most 7 monomers, at most 8 monomers, at most 9 monomers, at most 10 monomers, at most 11 monomers, at most 12 monomers, at most 13 monomers, at most 14 monomers, at most 15 monomers, at most 16 monomers, at most 17 monomers, at most 18 monomers, at most 19 monomers, at most 20 monomers, at most 21 monomers, at most 22 monomers, at most 23 monomers, at most 24 monomers, at most at least 25 monomers, at most 26 monomers, at most 27 monomers, at most 28 monomers, at most 29 monomers or at most 30 monomers. In some embodiments, the PEG polymer includes a terminal OCH₃ instead of a terminal OH. In some embodiments, the PEG polymer includes a terminal OCH₂CH₃ instead of a terminal OH.

In still other aspects of this embodiment, the number of monomers comprising an activated PEG polymers useful to modify a compound disclosed herein can be, e.g., about 4 to about 8 monomers, about 4 to about 12 monomers, about 4 to about 16 monomers, about 4 to about 20 monomers, about 4 to about 24 monomers, about 4 to about 28 monomers, about 4 to about 32 monomers, about 6 to about 8 monomers, about 6 to about 12 monomers, about 6 to about 16 monomers, about 6 to about 20 monomers, about 6 to about 24 monomers, about 6 to about 28 monomers, about 6 to about 32 monomers, about 8 to about 12 monomers, about 8 to about 16 monomers, about 8 to about 20 monomers, about 8 to about 24 monomers, about 8 to about 28 monomers, about 8 to about 32 monomers, about 10 to about 12 monomers, about 10 to about 16 monomers, about 10 to about 20 monomers, about 10 to about 24 monomers, about 10 to about 28 monomers, about 10 to about 32 monomers, about 12 to about 16 monomers, about 12 to about 20 monomers, about 12 to about 24 monomers, about 12 to about 28 monomers, about 12 to about 32 monomers, about 14 to about 16 monomers, about 14 to about 20 monomers, about 14 to about 24 monomers, about 14 to about 28 monomers, about 14 to about 32 monomers, about 16 to about 20 monomers, about 16 to about 24 monomers, about 16 to about 28 monomers, about 16 to about 32 monomers, about 18 to about 20 monomers, about 18 to about 24 monomers, about 18 to about 28 monomers, about 18 to about 32 monomers, about 20 to about 24 monomers, about 20 to about 28 monomers, about 20 to about 32 monomers, about 22 to about 24 monomers, about 22 to about 28 monomers, about 22 to about 32 monomers, about 24 to about 28 monomers, about 24 to about 32 monomers, about 26 to about 28 monomers, about 26 to about 32 monomers, about 28 to about 32 monomers or about 30 to about 32 monomers. In some embodiments, the PEG polymer includes a terminal OCH₃ instead of a terminal OH. In some embodiments, the PEG polymer includes a terminal OCH₂CH₃ instead of a terminal OH.

In aspects of this embodiment, the molecular weight of the activated PEG polymer can be, e.g., about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, or about 100 kDa. In other aspects of this embodiment, the molecular weight of the activated PEG polymer derivative can be, e.g., at least 20 kDa, at least 25 kDa, at least 30 kDa, at least 35 kDa, at least 40 kDa, at least 45 kDa, at least 50 kDa, at least 55 kDa, at least 60 kDa, at least 65 kDa, at least 70 kDa, at least 75 kDa, at least 80 kDa, at least 85 kDa, at least 90 kDa, at least 95 kDa, or at least 100 kDa. In yet other aspects of this embodiment, the molecular weight of the activated PEG polymer derivative can be, e.g., at most 20 kDa, at most 25 kDa, at most 30 kDa, at most 35 kDa, at most 40 kDa, at most 45 kDa, at most 50 kDa, at most 55 kDa, at most 60 kDa, at most 65 kDa, at most 70 kDa, at most 75 kDa, at most 80 kDa, at most 85 kDa, at most 90 kDa, at most 95 kDa, or at most 100 kDa. In still other aspects of this embodiment, the molecular weight of the activated PEG polymer derivative can be, e.g., about 20 kDa to about 30 kDa, about 20 kDa to about 40 kDa, about 20 kDa to about 50 kDa, about 20 kDa to about 60 kDa, about 20 kDa to about 70 kDa, about 20 kDa to about 80 kDa, about 20 kDa to about 90 kDa, about 20 kDa to about 100 kDa, about 30 kDa to about 40 kDa, about 30 kDa to about 50 kDa, about 30 kDa to about 60 kDa, about 30 kDa to about 70 kDa, about 30 kDa to about 80 kDa, about 30 kDa to about 90 kDa, about 30 kDa to about 100 kDa, about 40 kDa to about 50 kDa, about 40 kDa to about 60 kDa, about 40 kDa to about 70 kDa, about 40 kDa to about 80 kDa, about 40 kDa to about 90 kDa, about 40 kDa to about 100 kDa, about 50 kDa to about 60 kDa, about 50 kDa to about 70 kDa, about 50 kDa to about 80 kDa, about 50 kDa to about 90 kDa, about 50 kDa to about 100 kDa, about 60 kDa to about 70 kDa, about 60 kDa to about 80 kDa, about 60 kDa to about 90 kDa, about 60 kDa to about 100 kDa, about 70 kDa to about 80 kDa, about 70 kDa to about 90 kDa, about 70 kDa to about 100 kDa, about 80 kDa to about 90 kDa, about 80 kDa to about 100 kDa, or about 90 kDa to about 100 kDa. In some embodiments, the PEG polymer includes a terminal OCH₃ instead of a terminal OH. In some embodiments, the PEG polymer includes a terminal OCH₂CH₃ instead of a terminal OH.

In aspects of this embodiment, the molecular weight of the PEG-conjugated compound can be, e.g., about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, or about 100 kDa. In other aspects of this embodiment, the molecular weight of the PEG-conjugated compound can be, e.g., at least 20 kDa, at least 25 kDa, at least 30 kDa, at least 35 kDa, at least 40 kDa, at least 45 kDa, at least 50 kDa, at least 55 kDa, at least 60 kDa, at least 65 kDa, at least 70 kDa, at least 75 kDa, at least 80 kDa, at least 85 kDa, at least 90 kDa, at least 95 kDa, or at least 100 kDa. In yet other aspects of this embodiment, the molecular weight of the PEG-conjugated compound can be, e.g., at most 20 kDa, at most 25 kDa, at most 30 kDa, at most 35 kDa, at most 40 kDa, at most 45 kDa, at most 50 kDa, at most 55 kDa, at most 60 kDa, at most 65 kDa, at most 70 kDa, at most 75 kDa, at most 80 kDa, at most 85 kDa, at most 90 kDa, at most 95 kDa, or at most 100 kDa. In still other aspects of this embodiment, the molecular weight of the PEG-conjugated compound can be, e.g., about 20 kDa to about 30 kDa, about 20 kDa to about 40 kDa, about 20 kDa to about 50 kDa, about 20 kDa to about 60 kDa, about 20 kDa to about 70 kDa, about 20 kDa to about 80 kDa, about 20 kDa to about 90 kDa, about 20 kDa to about 100 kDa, about 30 kDa to about 40 kDa, about 30 kDa to about 50 kDa, about 30 kDa to about 60 kDa, about 30 kDa to about 70 kDa, about 30 kDa to about 80 kDa, about 30 kDa to about 90 kDa, about 30 kDa to about 100 kDa, about 40 kDa to about 50 kDa, about 40 kDa to about 60 kDa, about 40 kDa to about 70 kDa, about 40 kDa to about 80 kDa, about 40 kDa to about 90 kDa, about 40 kDa to about 100 kDa, about 50 kDa to about 60 kDa, about 50 kDa to about 70 kDa, about 50 kDa to about 80 kDa, about 50 kDa to about 90 kDa, about 50 kDa to about 100 kDa, about 60 kDa to about 70 kDa, about 60 kDa to about 80 kDa, about 60 kDa to about 90 kDa, about 60 kDa to about 100 kDa, about 70 kDa to about 80 kDa, about 70 kDa to about 90 kDa, about 70 kDa to about 100 kDa, about 80 kDa to about 90 kDa, about 80 kDa to about 100 kDa, or about 90 kDa to about 100 kDa. In some embodiments, the PEG polymer includes a terminal OCH₃ instead of a terminal OH. In some embodiments, the PEG polymer includes a terminal OCH₂CH₃ instead of a terminal OH.

Aspects of the present specification disclose, in part, a EphA4 antagonist that exhibits physiological stability. Physiological stability includes properties such as, e.g., biological half-life and plasma half-life. A biological half-life is the time required for one half of the total amount of a particular substance in a biological system to be degraded or eliminated by biological processes such as, e.g., through the kidney, liver and excretion functions when the rate of removal is nearly exponential. Typically, a biological half-life is measured by assaying a pharmacologic and/or physiologic property of the substance. A plasma half-life is the time required for one half of the total concentration of a particular substance in a biological system to reach its steady-state value in blood plasma. The relationship between the biological and plasma half-lives of a substance can be complex, due to factors including accumulation in tissues, active metabolites, and receptor interactions.

In an embodiment, an EphA4 antagonist can exhibit a therapeutically effective biological half-life. In aspects of this embodiment, a compound disclosed herein, can exhibit a biological half-life of, e.g., about 12 hours, about 18 hours, about 24 hours, about 30 hours, about 36 hours, about 42 hours, about 48 hours, about 54 hours, about 60 hours, about 66 hours, about 72 hours, about 78 hours, about 84 hours, about 90 hours or about 96 hours. In still other aspects of this embodiment, compound disclosed herein, can exhibit a biological half-life of, e.g., 12 hours to 24 hours, 12 hours to 36 hours, 12 hours to 48 hours, or 12 hours to 60 hours.

In another embodiment, an EphA4 antagonist can exhibit a therapeutically effective plasma half-life. In aspects of this embodiment, a compound disclosed herein, can exhibit a plasma half-life of, e.g., about 30 min, about 1 hour, about 2 hours, about 4 hours, about 8 hours, about 12 hours, about 18 hours, about 24 hours, about 30 hours, about 36 hours, about 42 hours, about 48 hours, about 54 hours, about 60 hours, about 66 hours, about 72 hours, about 78 hours, about 84 hours, about 90 hours or about 96 hours, or a plasma half-life within a range defined by any of the preceding values. In other aspects of this embodiment, a compound disclosed herein, can exhibit a plasma half-life of, e.g., at least 30 min, at least 1 hour, at least 2 hours, at least 4 hours, at least 8 hours, at least 12 hours, at least 18 hours, at least 24 hours, at least 30 hours, at least 36 hours, at least 42 hours, at least 48 hours, at least 54 hours, at least 60 hours, at least 66 hours, at least 72 hours, at least 78 hours, at least 84 hours, at least 90 hours or at least 96 hours.

Aspects of the present specification disclose, in part, a pharmaceutical composition. As used herein, the term “pharmaceutical composition” is synonymous with “therapeutic composition” or “pharmaceutically acceptable therapeutic composition” and refers to a composition comprising a therapeutically effective concentration of an active ingredient, such as, e.g., a EphA4 antagonist as disclosed herein. A pharmaceutical composition disclosed herein can comprise a single EphA4 antagonist as disclosed herein. Alternatively, a pharmaceutical composition disclosed herein can comprise a plurality of EphA4 antagonists as disclosed herein. In aspects of this embodiment, pharmaceutical composition disclosed herein can comprise about one, about two, about three, about four, or about five EphA4 antagonists as disclosed herein. In other aspects of this embodiment, pharmaceutical composition disclosed herein can comprise one or more, two or more, three or more, four or more or five or more EphA4 antagonists as disclosed herein. In yet other aspects of this embodiment, pharmaceutical composition disclosed herein can comprise at most one, at most two, at most three, at most four, or at most five EphA4 antagonists as disclosed herein. In still other aspects of this embodiment, pharmaceutical composition disclosed herein can comprise about one to about two, about one to about three, about one to about four, about one to about five, about two to about three, about two to about four, about two to about five, about three to about four, about three to about five or about four to about five, EphA4 antagonists.

The amount of EphA4 antagonist included in a pharmaceutical composition is an amount sufficient to elicit an appropriate therapeutic response in the individual. Typically, this amount is also one that does not cause significant adverse side effects. Such amount will vary depending on which specific EphA4 antagonist(s) are employed. An optimal amount for a particular pharmaceutical composition can be ascertained by standard studies involving observation of antibody titers and other responses in individuals.

Generally, an effective and safe amount of EphA4 antagonist, like a compound disclosed herein, included in a pharmaceutical composition varies from about 1 ng to 1,000 μg. In aspects of this embodiment, an amount of a compound disclosed herein, included in a therapeutic composition can be, e.g., about 1 ng, about 2 ng, about 3 ng, about 4 ng, about 5 ng, about 6 ng, about 7 ng, about 8 ng, about 9 ng, about 10 ng, about 15 ng, about 20 ng, about 25 ng, about 30 ng, about 35 ng, about 40 ng, about 45 ng, about 50 ng, about 55 ng, about 60 ng, about 65 ng, about 70 ng, about 75 ng, about 80 ng, about 85 ng, about 90 ng, about 95 ng, about 100 ng, about 110 ng, about 120 ng, about 130 ng, about 140 ng, about 150 ng, about 160 ng, about 170 ng, about 180 ng, about 190 ng, about 200 ng, about 210 ng, about 220 ng, about 230 ng, about 240 ng, about 250 ng, 260 ng, about 270 ng, about 280 ng, about 290 ng, about 300 ng, about 310 ng, about 320 ng, about 330 ng, about 340 ng, about 350 ng, 360 ng, about 370 ng, about 380 ng, about 390 ng, about 400 ng, about 410 ng, about 420 ng, about 430 ng, about 440 ng, about 450 ng, 460 ng, about 470 ng, about 480 ng, about 490 ng, about 500 ng, about 510 ng, about 520 ng, about 530 ng, about 540 ng, about 550 ng, 560 ng, about 570 ng, about 580 ng, about 590 ng, about 600 ng, about 610 ng, about 620 ng, about 630 ng, about 640 ng, about 650 ng, 660 ng, about 670 ng, about 680 ng, about 690 ng, about 700 ng, about 710 ng, about 720 ng, about 730 ng, about 740 ng, about 750 ng, 760 ng, about 770 ng, about 780 ng, about 790 ng, about 800 ng, about 810 ng, about 820 ng, about 830 ng, about 840 ng, about 850 ng, 860 ng, about 870 ng, about 880 ng, about 890 ng, about 900 ng, about 910 ng, about 920 ng, about 930 ng, about 940 ng, about 950 ng, 960 ng, about 970 ng, about 980 ng, about 990 ng, or about 1,000 ng, or within a range defined by any of the preceding values.

A pharmaceutical composition disclosed herein can optionally include one or more pharmaceutically acceptable carriers that facilitate processing of an active ingredient into therapeutic compositions. As used herein “pharmaceutically acceptable” refers to any molecular entity or composition that does not produce an adverse, allergic or other untoward or unwanted reaction when administered to an individual. As used herein, the term “pharmacologically acceptable carriers” is synonymous with “pharmacological carriers” and means any compound that has substantially no long term or permanent detrimental effect when administered and encompasses terms such as “pharmacologically acceptable vehicle, stabilizer, diluent, additive, auxiliary or excipient.” Such a carrier generally is mixed with an active compound, or permitted to dilute or enclose the active compound and can be a solid, semi-solid, or liquid agent. It is understood that the active ingredients can be soluble or can be delivered as a suspension in the desired carriers. Any of a variety of pharmaceutically acceptable carrier can be used including, without limitation, aqueous media such as, e.g., water, saline, and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN'S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003).

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a therapeutic composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. An active ingredient, such as, e.g., a compound disclosed herein, can be provided as a salt.

A pharmaceutical composition can be administered to an individual alone, or in combination with other supplementary active ingredients, agents, drugs or hormones. The pharmaceutical compositions can be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

Aspects of the present specification disclose, in part, a method of treating an EphA4-based disease, disorder or pathology. An EphA4-based disease, disorder or pathology refers to any condition, disease or disorder or pathology where a pathophysiology effect is due to dysregulation of EphA4 signaling in a manner that causes EphA4 signaling hyperactivity in cells or spatially or temporally aberrant EphA4 signaling.

Such methods include therapeutic (following onset of an EphA4-based disease) and prophylactic (prior to onset of an EphA4-based disease). For example, therapeutic and prophylactic methods of treating an individual for an EphA4-based disease, disorder or pathology include treating an individual at risk of having an EphA4-based disease, disorder or pathology, treating an individual having an EphA4-based disease, disorder or pathology, and methods of protecting an individual from an EphA4-based disease, disorder or pathology, to decrease or reduce the probability of an EphA4-based disease, disorder or pathology in an individual, to decrease or reduce susceptibility of an individual to an EphA4-based disease, disorder or pathology, or to inhibit or prevent an EphA4-based disease, disorder or pathology in an individual, and to decrease, reduce, inhibit or suppress transmission of an EphA4-based disease, disorder or pathology from an afflicted individual to an unafflicted individual. Such methods include administering a pharmaceutical composition disclosed herein to therapeutically or prophylactically treat an individual having or at risk of having an EphA4-based disease, disorder or pathology. Accordingly, methods can treat an EphA4-based disease or pathology, or provide the individual with protection from an EphA4-based disease, disorder or pathology (e.g., prophylactic protection).

In an embodiment, a method of treating an EphA4-based disease, disorder or pathology, comprises administering one or more EphA4 antagonists, like one or more compounds disclosed herein, or a pharmaceutical composition disclosed herein to an individual in need thereof in an amount sufficient to reduce one or more physiological conditions or symptom associated with an EphA4-based disease, disorder or pathology, thereby treating the EphA4-based disease, disorder or pathology. In aspects of this embodiment, an EphA4-based disease, disorder or pathology includes, without limitation, a neurodegenerative disease, a hearing loss, promotion of nerve regeneration, promotion of neuroprotection, and a cancer.

Neurodegenerative diseases are conditions that affect brain or peripheral nerve function. They result from the deterioration of neurons and they are characterized by progressive central or peripheral nervous dysfunction. They are divided into two groups: conditions causing problems with movement or sensation and conditions affecting memory or related to dementia. EphA4 signaling activity has important functions in both categories. For example, increased expression of EphA4 and its activation by ephrin ligands contribute to the pathogenesis of ALS, Alzheimer's disease, multiple sclerosis, stroke and traumatic brain injury and other neurodegenerative disease because EphA4 signaling leads to abnormal inhibition of axon growth, aberrant synaptic function and poor neuronal survival. Thus, a compound disclosed herein, or a pharmaceutical composition disclosed herein can be useful in treating any neurodegenerative disease expressing high EphA4 levels. A neurodegenerative disease includes, without limitation, an Alexander disease, an Alper's disease, Alzheimer's disease, an amyotrophic lateral sclerosis, an ataxia telangiectasia, a Canavan disease, a Cockayne syndrome, a corticobasal degeneration, a Creutzfeldt-Jakob disease, a Guillain-Barre Syndrome a HIV-induced neurodegeneration, a Huntington disease, a Kennedy's disease, a Krabbe disease, a Lewy body dementia, a Machado-Joseph disease, a multiple sclerosis, a Parkinson's disease, a Pelizaeus-Merzbacher disease, a Pick's disease, a primary lateral sclerosis, a Refsum's disease, a Sandhoff disease, a Schilder's disease, a spinal cord injury, a Steele-Richardson-Olszewski disease, a stroke, a tabes dorsalis and/or a traumatic brain injury. Symptoms associated with a neurodegenerative disease include, without limitation, abnormal movement, abnormal sensation, limb grasping, muscle weakness, atrophy, paralysis, abnormal inhibition of axon growth, abnormal axonal transport, aberrant synaptic function, synaptic transmission loss, impaired synaptic plasticity, synaptic loss, neuronal degeneration, motor neuron degeneration, motor neuron loss, poor neuronal survival, memory loss, impaired learning, dementia, β-amyloid plaque deposits, aberrant neurofilament accumulation, reactive astroglia and/or reactive microglia.

In another embodiment, a method of treating an EphA4-based disease, disorder or pathology includes a method of treating a neurodegenerative disease. In an aspect of this embodiment, a method of treating a neurodegenerative disease comprises administering one or more compounds disclosed herein, or a pharmaceutical composition disclosed herein to an individual in need thereof in an amount sufficient to reduce one or more physiological conditions or symptom associated with a neurodegenerative disease, thereby treating the neurodegenerative disease.

A compound disclosed herein, or a pharmaceutical composition disclosed herein can also be administered to an individual in combination with other therapeutic compounds to increase the overall therapeutic effect of the treatment. The use of multiple compounds to treat an indication can increase the beneficial effects while reducing the presence of side effects.

As used herein, the term “amino acid” or “amino acids” means naturally occurring L-α-amino acids as well as non-naturally occurring D- and L-α-amino acids and commercially available synthetic amino acids include amino acids wherein the amino group is separated from the carboxyl group by more than one carbon atom such as β-alanine, γ-aminobutyric acid, 6-aminohexanoic acid and the like.

Names, three letter and one letter abbreviations of select amino acids are provided in the following table.

| Name | Abbr (3 letter) | Abbr (1 letter) |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Hydroxyproline | Hyp | O |
| Isoleucine | Ile | I |
| Leucine | Leu | L |
| Lysine | Lys | K |
| Methionine | Met | M |
| Phenylalanine | Phe | F |
| Proline | Pro | P |
| Pyroglutamatic | Glp | U |
| Serine | Ser | S |
| Threonine | Thr | T |
| Tryptophan | Trp | W |
| Tyrosine | Tyr | Y |
| Valine | Val | V |
| 6-Aminohexanoic acid | Ahx | |
| β-Alanine | β-Ala | βA |

Aspects of the present specification can also be described as follows:

An EphA4 receptor antagonist comprising a chemical entity having the structure of Formula I, βAPYCVY-Z1-βASWSCZ2 (SEQ ID NO: 26), or a pharmaceutically acceptable salt thereof, wherein Z¹ can be absent or selected from the group consisting of R, K, K(octanoyl), K(lauroyl), K(lauryl-G), K(lauroyl-βD), K(lauroyl-γE), and K(palmitoyl-γΣ); Z² is absent or selected from the group consisting of NH₂, GGRG-NH₂ (SEQ ID NO: 27), GGK(lauroyl)G-NH₂ (SEQ ID NO: 28), GGK(lauroyl-βD)G-NH₂ (SEQ ID NO: 29), GGK(lauroyl-γE)G-NH₂ (SEQ ID NO: 30), GGK(lauroyl-γEγE)G-NH₂ (SEQ ID NO: 23), GGK(myristoyl-γEγE)G-NH₂ (SEQ ID NO: 31), GGK(palmitoyl-γE)G-NH₂ (SEQ ID NO: 32), GGK(palmitoyl-γEγE)G-NH₂ (SEQ ID NO: 33), GGGSK(palmitoyl-γEγE)G-NH₂ (SEQ ID NO: 34), RLIEDICLPRWGCLWEDD-NH₂ (SEQ ID NO: 35), and L¹-PEG; L¹ is a linker; and Z¹ or Z¹ is not absent.

In some embodiments, Z¹ is selected from the group consisting of R, K, K(octanoyl), K(lauroyl-βD), and K(lauroyl-γE).

In some embodiments, Z¹ is selected from the group consisting of R, K, and K(octanoyl).

In some embodiments, Z¹ is R, or K.

In some embodiments, Z¹ is R.

In some embodiments, Z¹ is absent.

In some embodiments, Z² is selected from the group consisting of NH₂, GGRG-NH₂ (SEQ ID NO: 27), GGK(lauroyl)G-NH₂ (SEQ ID NO: 28), GGK(lauroyl-βD)G-NH₂ (SEQ ID NO: 29), GGK(lauroyl-γE)G-NH₂ (SEQ ID NO: 30), GGK(lauroyl-γEγE)G-NH₂ (SEQ ID NO: 23), GGK(myristoyl-γEγE)G-NH₂ (SEQ ID NO: 31), GGK(palmitoyl-γE)G-NH₂ (SEQ ID NO: 32), GGK(palmitoyl-γEγE)G-NH₂ (SEQ ID NO: 33), GGGSK(palmitoyl-γEγE)G-NH₂ (SEQ ID NO: 34), RLIEDICLPRWGCLWEDD-NH₂ (SEQ ID NO: 35), and L¹-PEG.

In some embodiments, Z² is selected from the group consisting of GGRG-NH₂ (SEQ ID NO: 27), GGK(lauroyl)G-NH₂ (SEQ ID NO: 28), GGK(lauroyl-βD)G-NH₂ (SEQ ID NO: 29), GGK(lauroyl-γE)G-NH₂ (SEQ ID NO: 29), GGK(lauroyl-γEγE)G-NH₂ (SEQ ID NO: 30), GGK(myristoyl-γEγE)G-NH₂ (SEQ ID NO: 23), GGK(palmitoyl-γE)G-NH₂ (SEQ ID NO: 31), GGK(palmitoyl-γEγE)G-NH₂ (SEQ ID NO: 33), GGGSK(palmitoyl-γEγE)G-NH₂ (SEQ ID NO: 34), RLIEDICLPRWGCLWEDD-NH₂ (SEQ ID NO: 35), and L¹-PEG.

In some embodiments, Z² is selected from the group consisting of GGK(lauroyl-βD)G-NH₂ (SEQ ID NO: 29), GGK(lauroyl-γE)G-NH₂ (SEQ ID NO: 30), GGK(lauroyl-γEγE)G-NH₂ (SEQ ID NO: 23), GGK(myristoyl-γEγE)G-NH₂ (SEQ ID NO: 31), GGK(palmitoyl-γE)G-NH₂ (SEQ ID NO: 32), GGK(palmitoyl-γEγE)G-NH₂ (SEQ ID NO: 33), GGGSK(palmitoyl-γEγE)G-NH₂ (SEQ ID NO: 34), RLIEDICLPRWGCLWEDD-NH₂ (SEQ ID NO: 35), and L¹-PEG.

In some embodiments, Z² is selected from the group consisting of GGK(lauroyl-γEγE)G-NH₂ (SEQ ID NO: 23), GGK(myristoyl-γEγE)G-NH₂ (SEQ ID NO: 31), GGGSK(palmitoyl-γEγE)G-NH₂ (SEQ ID NO: 34), RLIEDICLPRWGCLWEDD-NH₂ (SEQ ID NO: 35), and L¹-PEG.

In some embodiments, Z² is selected from the group consisting of GGK(lauroyl-γEγE)G-NH₂ (SEQ ID NO: 23), RLIEDICLPRWGCLWEDD-NH₂ (SEQ ID NO: 35), and L¹-PEG.

In some embodiments, Z² is L¹-PEG. In some embodiments, the PEG includes a terminal OCH₃ instead of a terminal OH. In some embodiments, the PEG includes a terminal OCH₂CH₃ instead of a terminal OH.

In some embodiments, L¹ is

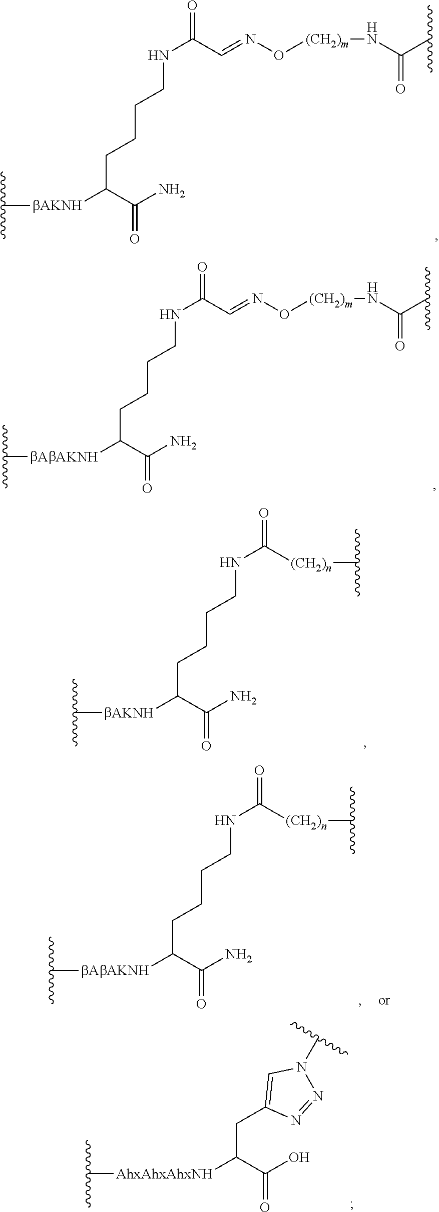

m is 1 to 5; and n is 0 to 5.

In some embodiments, L¹ is a peptide of at least four amino acids in length; and the peptide does not include a C (Cys).

In some embodiments, L¹ is a peptide of no greater than ten amino acids in length.

In some embodiments, L¹ is a tetrapeptide, pentapeptide or hexapeptide.

In some embodiments, L¹ is a tetrapeptide; and the tetrapeptide is GGKG (SEQ ID NO: 24).

In some embodiments, PEG is OCH₂CH₂(OCH₂CH₂)ₛOH, OCH₂CH₂(OCH₂CH₂)ₛOCH₃ or OCH₂CH₂(OCH₂CH₂)ₛOCH₂CH₃; and s is 500 to 1000.

In some embodiments, the chemical entity is any one of compounds 3 to 21, APY-d3-PEG1, APY-d3-PEG2, APY-d3-PEG3, and APY-d3-PEG4 of Table 1, or a pharmaceutically acceptable salt thereof.

In some embodiments, the chemical entity has a cyclic structure and the two C (Cys) taken together have a disulfide bond.

In some embodiments, the modified EphA4 receptor antagonist has an equilibrium disassociation rate constant for an EphA4 receptor less than 500 nM, less than 450 nM, less than 400 nM, less than 350 nM, less than 300 nM, less than 250 nM, less than 200 nM, less than 150 nM, less than 100 nM, less than 75 nM, less than 50 nM, less than 25 nM, less than 20 nM, less than 15 nM, less than 10 nM, less than 5 nM, less than 1 nM, less than 0.5 nM or less than 0.1 nM.

In some embodiments, the EphA4 receptor antagonist has an equilibrium disassociation rate constant for an EphA4 receptor of between about 1 nM to about 10 nM, about 1 nM to about 25 nM, about 1 nM to about 75 nM, about 1 nM to about 100 nM, about 1 nM to about 125 nM, about 1 nM to about 150 nM, about 5 nM to about 10 nM, about 5 nM to about 25 nM, about 5 nM to about 75 nM, about 5 nM to about 100 nM, about 5 nM to about 125 nM, about 5 nM to about 150 nM, about 10 nM to about 25 nM, about 10 nM to about 50 nM, about 10 nM to about 75 nM, about 10 nM to about 100 nM, about 10 nM to about 125 nM, about 10 nM to about 150 nM, about 10 nM to about 175 nM or about 10 nM to about 200 nM.

In some embodiments, the EphA4 receptor antagonist has an equilibrium disassociation rate constant for an ephrin-binding pocket of an ephrin receptor other than an EphA4 receptor of at least 2-fold more, at least 3-fold more, at least 4-fold more, at least 5-fold more, at least 6-fold more, at least 7-fold more, at least 8-fold more, or at least 9-fold more, at least 10-fold more, at least 20-fold more, at least 30-fold more, at least 40-fold more, at least 50-fold more, at least 60-fold more, at least 70-fold more, at least 80-fold more, at least 90-fold more, at least 100-fold more, at least 200-fold more, at least 300-fold more, at least 400-fold more, at least 500-fold more, at least 600-fold more, at least 700-fold more, at least 800-fold more, at least 900-fold more, at least 1,000-fold more, at least 2,500-fold more, at least 5,000-fold more, at least 7,500-fold more or at least 10,000-fold more.

In some embodiments, the EphA4 receptor antagonist reduces EphA4 receptor activity by at least 10%, at least 20%, at least 30%, at least 40%, at least 50%, at least 60%, at least 70%, at least 80%, at least 60%, or at least 100%.

In some embodiments, the EphA4 receptor antagonist has a plasma half-life of at least 6 hours, at least 12 hours, at least 18 hours, at least 24 hours, at least 30 hours, at least 36 hours, at least 42 hours, at least 48 hours, at least 54 hours, at least 60 hours, at least 66 hours, at least 72 hours, at least 78 hours, at least 84 hours, at least 90 hours or at least 96 hours.

In some embodiments, the EphA4 receptor antagonist has a plasma half-life of about 6 hours to about 24 hours, about 12 hours to about 36 hours, about 12 hours to about 48 hours, about 12 hours to about 60 hours, about 12 hours to about 72 hours, about 12 hours to about 84 hours, about 12 hours to about 96 hours, about 24 hours to about 36 hours, about 24 hours to about 48 hours, about 24 hours to about 60 hours, about 24 hours to about 72 hours, about 24 hours to about 84 hours, about 24 hours to about 96 hours, about 36 hours to about 48 hours, about 36 hours to about 60 hours, about 36 hours to about 72 hours, about 36 hours to about 84 hours, about 36 hours to about 96 hours, about 48 hours to about 60 hours, about 48 hours to about 72 hours, about 48 hours to about 84 hours, about 48 hours to about 96 hours, about 60 hours to about 72 hours, about 60 hours to about 84 hours, about 60 hours to about 96 hours, about 72 hours to about 84 hours, about 72 hours to about 96 hours or about 84 hours to about 96 hours.

Some embodiments provide a pharmaceutical composition comprising a EphA4 receptor antagonist.

In some embodiments of the pharmaceutical composition, the EphA4 receptor antagonist is further present in an amount of between 95 ng to 1,005 μg.

In some embodiments of the pharmaceutical composition, the pharmaceutical composition further comprises one or more pharmaceutical acceptable carriers.

Some embodiments provide use of a EphA4 receptor antagonist or a pharmaceutical composition in the treatment of an EphA4-based disease, disorder or pathology.

Some embodiments provide use of a EphA4 receptor antagonist or a pharmaceutical composition in the manufacture of a medicament for treating an EphA4-based disease, disorder or pathology.

Some embodiments provide a method of treating an EphA4-based disease, disorder or pathology, the method comprising administering a EphA4 receptor antagonist or a pharmaceutical composition to an individual in need thereof, wherein administration reduces one or more symptoms associated with the EphA4-based disease, disorder or pathology.

In some embodiments, the EphA4-based disease, disorder or pathology comprises a condition, a disease, a disorder and/or pathology where a pathophysiology effect is due to dysregulation of EphA4 signaling in a manner that causes EphA4 signaling hyperactivity in cells or spatially or temporally aberrant EphA4 signaling.

In some embodiments, the EphA4-based disease, disorder or pathology is a neurodegenerative disease, a hearing loss, a promotion of nerve regeneration, a promotion of neuroprotection, or a cancer.

In some embodiments, the neurodegenerative disease is an Alexander disease, an Alper's disease, Alzheimer's disease, an amyotrophic lateral sclerosis, an ataxia telangiectasia, a Canavan disease, a Cockayne syndrome, a corticobasal degeneration, a Creutzfeldt-Jakob disease, a Guillain-Barre Syndrome a HIV-induced neurodegeneration, a Huntington disease, a Kennedy's disease, a Krabbe disease, a Lewy body dementia, a Machado-Joseph disease, a multiple sclerosis, a Parkinson's disease, a Pelizaeus-Merzbacher disease, a Pick's disease, a primary lateral sclerosis, a Refsum's disease, a Sandhoff disease, a Schilder's disease, a spinal cord injury, a Steele-Richardson-Olszewski disease, a stroke, a tabes dorsalis and/or a traumatic brain injury.

In some embodiments, the one or more symptoms include abnormal movement, abnormal sensation, limb grasping, muscle weakness, atrophy, paralysis, abnormal inhibition of axon growth, abnormal axonal transport, aberrant synaptic function, synaptic transmission loss, impaired synaptic plasticity, synaptic loss, neuronal degeneration, motor neuron degeneration, motor neuron loss, poor neuronal survival, memory loss, impaired learning, dementia, β-amyloid plaque deposits, aberrant neurofilament accumulation, reactive astroglia and/or reactive microglia.

In some embodiments, the cancer is a glioblastoma, a gastric cancer, a pancreatic cancer, a prostate cancer, a breast cancer, a liver cancer, a leukemia, a melanoma, a lung cancer or a Sezary syndrome.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, pharmaceutical compositions, or methods or uses of treating a disorder disclosed herein.

### Example 1

### Synthesis of unmodified peptides, lipidated APY-d3 derivatives (peptides 4-20), and peptide 21

APY-d2, APY-d3, peptides 1-3 and peptides to be coupled to lipids or PEG were synthesized using manual synthetic cycles for Fmoc solid phase synthesis. Typically, those syntheses were performed on a 0.2 mmol scale using either Rink amide aminomethyl resin (0.69 mmol/g, Novabiochem; e.g., Catalogue #855130 or Catalogue #855004, EMD Millipore Corporation, Burlington, USA) or Rink amide ChemMatrix resin (0.4 mmol/g, Biotage) using standard Fmoc side chain protecting groups (Tyr/Ser, tBu; Cys, Trt; Arg, Pbf; Trp, Boc) unless otherwise noted. The bicyclic peptide 21 was synthesized with Acm protection on Cys4 and Cys12 and with Trt protection on Cys21 and Cys27. Couplings were performed with Fmoc protected amino acids (1.1 mmol) and neat N,N-diisopropylethylamine (DIEA, 1.5 mmol) dissolved in 2.5 mL of 0.4 M HCTU in DMF (1.0 mmol) for 20 min. The final residue (βA1a1) was coupled as Boc-βA1a-OH, except for precursor 1 used to generate APY-d3-PEG1 and APY-d3-PEG2, where βA1a1 was coupled as Fmoc-βA1a-OH (precursor 1: Fmoc-βAPYCVYRβASWSCβAβAK-NH₂ (SEQ ID NO: 36)). Fmoc deprotection was facilitated with 20% 4-methylpiperidine in DMF for a total of 7 min. After removal of the Fmoc group, N-terminal protection of precursor 1 was performed with 2-acetyldimedone (2.0 mmol) at 1.0 M in DMF. Protection was complete after 3 hours as indicated by qualitative ninhydrin test.

Acyl and bromoacetyl group coupling to Lys side chains was accomplished using orthogonal protection of the Lys side chain with ivDde (Fmoc-Lys (ivDde)-OH, Catalogue #852082, EMD Millipore Corporation, Burlington, USA). After primary chain elongation was completed, ivDde was removed from the Lys side chain by exposing the peptide to 4% hydrazine (8% hydrazine hydrate in DMF) in 4×5 min increments. γGlu couplings to the Lys side chain were performed as described above using Fmoc-Glu (OH)-OtBu (CAS No. 84793-07-7; Catalogue #852035, EMD Millipore Corporation, Burlington, USA). Acyl groups were coupled to the Lys &-amine using the corresponding acid chloride (2.0 mmol) at 1.0 M in DMF for 10 min. Bromoacetate coupling was achieved by pre-activating bromoacetic acid (1.0 mmol) with NHS (1.0 mmol) and neat DIC (1.0 mmol) for 45 min in DMF and adding the resultant mixture to the peptide-resin for 10 min. The secondary amine was formed by adding 1-octylamine or 1-dodecylamine (1.0 mmol) at 1.0 M in DMF to Lys-&-bromoacetate peptide-resin for 30 min.

Following all couplings, APY-d2, APY-d3 and peptides 1-14, 17, 18 and 21 were deprotected and cleaved from the resin with TFA:TIS:EDT:H₂O (37:1:1:1 v/v) for 2 hours at room temperature. The TFA was evaporated under N₂. Peptide was precipitated with ice-cold Et₂O, filtered and washed with Et₂O. The crude peptides were dissolved in 10% acetic acid, 45% acetonitrile in water and lyophilized. Samples were analyzed by reversed-phase high performance liquid chromatography (HPLC) and electrospray ionization mass spectrometry (ESI-MS). Peptides with significant side products were purified by HPLC.

Oxidation to form the disulfide bond was achieved by dissolving the peptides at 0.1 mg/ml in 1:1 acetonitrile: 0.2 M NH₄HCO₃, pH 8 and stirring overnight open to air. The oxidized peptide was purified by HPLC. For peptide 21, the first disulfide bond was formed by dissolving the peptide at 0.1 mg/ml in 0.1 M NH₄HCO₃, pH 8 and stirring overnight open to air, and purified by HPLC. The second disulfide was formed by redissolving the peptide at 0.3 mg/mL in 1:1 acetonitrile: water, 0.1% TFA with approximately 2 equivalents I₂ dissolved at 1.0 M in 1:1 acetonitrile: water, 0.1% TFA for 15 min. The reaction was quenched with sodium ascorbate and the peptide was purified by HPLC. For peptides 15, 16, 19, and 20 and precursor 1, the disulfide bond was formed on the resin. Fmoc-Cys (Acm)-OH was introduced at position 4 and, following chain elongation, the resin was equilibrated in MeOH:CH₂Cl₂:H₂O (60:25:4 v/v), 50 ml per mmol of peptide-resin (20 mM resin-bound peptide). To this mixture, an equal volume of 80 mM I₂ in CH₂Cl₂:MeOH (8:1.5 v/v) was added for 15 min at room temperature. The I₂ solution was drained and the remaining reagent was quenched with saturated sodium ascorbate: DMF (1:5 v/v) until no color remained. The peptides were then deprotected and cleaved from the resin in TFA:TIS:H₂O (38:1:1 v/v) and purified by HPLC as described above.

APY-d2, APY-d3, peptides 1-21 and precursor 1 were purified via semi-preparative HPLC on a Waters Prep LC 4000 system with a Jupiter 10 μm Proteo 90 Å (250×21.2 mm) column (Phenomenex) at flow rates of 15 mL/min using linear gradients of 0.5% buffer B/min (buffer A: 0.05% TFA in H₂O; buffer B: 0.045% TFA in 9:1 CH₃CN:H₂O). The molecular weight of each purified peptide was determined by ESI-MS (API 2000, PE/Sciex). Purity was determined by analytical reversed-phase HPLC and integration of the signal at 214 nm. Analytical HPLC was performed on either a Dynamax Rainin SD-200 system or an Agilent 1100 system using linear gradients of 0.5% buffer B/min on a Jupiter 4 μm Proteo 90 Å (150×4.6 mm) column (Phenomenex) at flow rates of 1 ml/min. Peptides were obtained at a non-optimized yield of 5-20%. Final purities were >90% for peptides.

The lipidated peptide 15 [Cyc(4,12)] H₂N-(bA)PYCVYR(bA)SWSCGG(K/Lauroyl)(γE)(γE))G-amide (SEQ ID NO: 37) was also synthesized according to the following protocol. The peptide base sequence was synthesized at a 0.25 mmole scale using Gyros Rink amide resin (0.35 mmol/g) on a CEM Liberty Blue instrument. Standard protecting groups were used for all amino acids, except for Lys, for which Fmoc-Lys (ivDde)-OH was used. All amino acids were coupled at a 5-fold excess using DIC/Oxyma activators; the first 8 amino acids were single coupled, while the remaining amino acids were double coupled. Upon sequence completion, the N-terminal Fmoc was removed, and the peptide was capped with a Boc protecting group. The Lys (ivDde) was deprotected using 2% hydrazine in DMF (2×15 min). After rinsing, both γGlu residues were coupled using 6 equivalents of γGlu (e.g., CAS No. 84793-07-7)/PyAOP/HOAt and 12 equivalents of DIPEA for 30 min at 40° C. Lauric acid was coupled at 40° C. overnight, using 6 equivalents of HBTU and 12 equivalents of DIPEA. Cleavage and global deprotection was performed at room temperature for 3 hours, using trifluoroacetic acid (TFA)/water/thioanisole/EMS/EDT (20:1:1:1:1). The cleaved peptide was precipitated using ether, and the precipitated peptide was rinsed with ether to remove scavengers, after which it was lyophilized. The crude material was dissolved in DMSO and TEA was added to induce disulfide formation; the reaction was allowed to proceed overnight. LC/MS was used to confirm disulfide formation. The peptide was lyophilized and then purified by RP-HPLC (column: C18, 120 Å, 10 μm, 25×250 mm) using a gradient of 30-50% Buffer B over 60 min (Buffer A: 0.1% TFA in water, Buffer B: 0.08% TFA n acetonitrile). The peptide was analyzed by ultra-high performance liquid chromatography (UHPLC)/MS to verify purity and determine the molecular weight. Pure fractions were lyophilized to yield the desired product. The average calculated mw of the peptide was 2, 142.0 (average theoretical mw 2, 142.5) and its purity was >95%.

### Example 2

### Synthesis of PEGylated APY-d3 derivatives

To generate APY-d3-PEG1, precursor 1 was dissolved at 0.5 mM in 0.1 M borate buffer and 45 mg of 30 kDa methoxy-PEG succinimidyl carboxymethyl ester (JenKem Technology USA) were added to the dissolved peptide for 30 min. To generate APY-d3-PEG2, precursor 1 was dissolved at 0.5 mM in 0.1 M borate buffer and 45 mg of 30 kDa methoxy-PEG-(CH₂)₅COO—NHS (#ME-300HS, Sunbright, NOF America Corporation) were added to the dissolved peptide for 6 hours. The PEGylated peptides were purified by HPLC using a 2% per minute gradient. The Dde was removed by treating the peptides dissolved at 0.5 mM in milli-Q H₂O with hydrazine (0.1 M final concentration) for 45 min, and the peptides were then immediately purified by reversed phase HPLC.

APY-d3-PEG3 was generated by attaching 30 kDa PEG to APY-d3 without protecting its N-terminus by taking advantage of an oxime-based bioconjugation reaction using substituted anilines as nucleophilic catalysts. APY-d3-βA1a-glyoxyl was generated for conjugation with purchased 30 kDa PEG containing an amino-oxy reactive group (methoxy-PEG-CONH(CH₂)₂—ONH₂; #ME-300CA, Sunbright, NOF America Corporation). Briefly, pure peptide-glyoxyl (1 eq) and amino-oxy-PEG 30 kDa (1.1 eq) were dissolved to a concentration of 1 mM peptide in acetate buffer (25 mM pH 4.5). Aniline was then spiked into a concentration of 50 mM and the reaction mixture was allowed to stir for 2-6 hours. An aliquot of the reaction was used for HPLC analysis and once it was confirmed that the reaction had proceeded to completion (based on total consumption of the starting peptide), the reaction mixture was transferred to a 10 kDa dialysis bag. The crude reaction mixture was dialyzed against pure water for 48 hours to remove any salts, catalyst, and unreacted peptide that might still be in the reaction mixture and lyophilized.

The APY-d3 PEG4 peptide [Cyc (4,12)] H₂N-(βA)PYCVYR(βA)SWSC(Ahx)(Ahx)(Ahx)(Pra/mPEG30-triazole)-OH (SEQ ID NO: 38), was synthesized according to the following steps.

Solid phase synthesis. The H-βA1a-Pro-Tyr(tBu)-Cys(Trt)-Val-Tyr(tBu)-Arg (Pbf)-βA1a-Ser(tBu)-Trp(Boc)-Ser(tBu)-Cys(Trt)-Ahx-Ahx-Ahx-Pra-OH (SEQ ID NO: 39) peptide was synthesized manually at 0.5 mmol scale, using Fmoc/tBu chemistry and Wang resin as solid support. The first amino acid, Fmoc-Pra-OH, was loaded on the Wang resin using DMAP/DCI (sub: 0.60 mmol/g, Lot: 009166L). The coupling of the amino acids (3 eq) was carried out with DIC (3 eq), Oxyma (3 eq) and 20% DIPEA in DMF activation chemistry for 6 hours or overnight at room temperature. The Fmoc group was removed by treating the peptide resin with 20% piperidine/DMF (2×, 2 min and 20 min each). Start resin weight: 0.83 g; end resin weight: 1.6 g.

Deprotection and cleavage. The peptide resin was washed with DMF, IPA, DCM, and ether and dried under vacuum for 24 hours. Then, the peptide deprotection and cleavage from the resin was performed by treatment with 30 mL of TFA/triisopropylsilane/water/anisole/thioanisole/DODT (87.5/2.5/5.0/2.5/1.5/1) v/v¾) for 2 hours at room temperature. The TFA was partially evaporated, and the crude product was precipitated and washed three times with ice-cold ether, redissolved in 20% CH₃CN/H₂O and lyophilized using a VirTis Sentry 2.0 Lyophilizer with no lyophilization cycle (full vacuum) for 24 hours. Theoretical crude: 0.92 g; actual crude: 0.55 g; yield: 59.78%.

Disulfide bond formation (oxidation) and purification. The linear crude product (0.55 g) was purified using a reversed phase HPLC column (Luna C18(3), 100 A, 5.0 cm diameter, Phenomenex Inc., Torrance, USA). The gradient used to purify the peptide was 15% to 45% B in 75 min. Buffer A was 0.1% TFA in H₂O and Buffer B was 100% acetonitrile and the flow rate used was 100 mL/min. The wavelength used to detect the product was 220 nm. The product eluted around 21.7% B and the fractions were analyzed by mass spectrometry (ZEVO G2, Waters) and analytical HPLC (Shimadzu) using a C-18 column (4.5×250 mm, particle size 5 μm, 200 A, No: 1 19YA90009, YMC-Pack-ODS-A, YMC, Japan). Pure fractions were combined to yield pure pools. The pH of the solution was adjusted to 7.9 and 200 mL of H₂O₂ were added. After stirring the solution for 24 hours, the reaction was quenched by lowering the pH with 50% acetic acid. The solution was loaded into the HPLC column, and the cyclized product was purified as above. LC/MS was used to confirm disulfide formation.

Click reaction and purification. The oxidized product (1 eq, 24.1 mg, 0.0131 mmol) and methoxy-PEG-azide (2.1 eq, 828 mg, 0.0276 mmol, Creative Works, Cat. WXH01O1 7) were dissolved in 7 mL of degassed H₂O and 3.6 mL of degassed tert-butanol. Then, 1 g of copper powder was added, and the solution was stirred for 24 hours at room temperature. The completion of the reaction was monitored by HPLC. The solvents were removed by evaporation and the impurities were removed by dialysis using H₂O. Then, the PEGylated peptide was dissolved in 20% acetonitrile/H₂O and the solution was lyophilized. Yield: 665 mg. The average calculated molecular weight of the oxidized peptide before PEGylation was 1,837.87 as determined by mass spectrometry (average theoretical molecular weight 1,838.2) and its purity was >95%, as determined by HPLC.

For biological assays, peptide stock solutions of ˜10 mM were made in H₂O, PBS or DMSO, and the concentration was determined by measuring absorbance at 280 nm. It should be noted that since PEG does not absorb at this wavelength, free PEG remaining in the final preparations of the PEGylated peptides is not detected.

### Example 3

### Determination of IC₅₀ Values for Inhibition of EphA4-Ephrin-A5 Binding

To determine IC₅₀ values for inhibition of EphA4-ephrin-A5 interaction by the compounds, human EphA4 Fc (#CJ75, Novoprotein) or mouse EphA4 Fc (#641-A4, R&D Systems/BioTechne) at 1 μg/mL in TBST (150 mM NaCl, 50 mM Tris-HCl, pH 7.5, 0.01% Tween 20) were immobilized on protein A-coated 96-well plates (#15132, Pierce/Thermo Scientific) for 1 hour at room temperature. The plates were then washed 3 times with TBST and incubated for ˜1 hour at room temperature with 0.05 nM ephrin-A5 alkaline phosphatase (AP) and different compound concentrations in 40 μL of TBST/well. To remove unbound ephrin-A5 AP, the plates were washed 3 times with TBST and the amount of bound ephrin-A5 AP was quantified by adding 1 mg/mL of p-nitrophenyl phosphate substrate (#34045, Pierce/Thermo Scientific) diluted in SEAP buffer (105 mM diethanolamine, 0.5 mM MgCl₂, pH 9.8). After ˜1 hour incubation at 37° C., absorbance was measured at 405 nm, and the absorbance from wells coated with Fc alone was subtracted as background.

To assess the Eph receptor selectivity of the compounds, 1 μg/mL Eph receptor Fc fusion proteins were immobilized on protein A-coated wells and incubated with 0.05 nM ephrin-A5 AP (for EphA receptors) or ephrin-B2 AP (for EphB receptors) in the presence or absence of the compound.

### Example 4

### Determination of IC₅₀ Values in the Presence of HSA or BSA

To determine whether albumin binding to the lipidated compounds affects their ability to bind EphA4, the effects of delipidated human serum albumin (HSA) on the inhibitory potency of some of the compounds were examined. By comparing compounds tested in parallel in the presence and in the absence of 40 mg/mL HSA (which is the concentration present in the blood), it was found that HSA has only a very small effect on the potency of the non-lipidated compounds 1, 2 and APY-d3 examined as controls, as expected (FIG. 6A-B and Table 1). HSA decreased the potency of the octanoyl compounds 4, 5, 6 and 7 by 2-3 fold, suggesting that the binding of serum albumin to these compounds interferes with EphA4 binding (FIG. 6A-B and Table 1). In contrast, HSA dramatically decreased the potency of the lauroyl compound 15 and the myristoyl compound 16, which can be impacted by higher HSA binding affinity of C12 and C14 lipids. The effects of HSA on the palmitoylated compounds were not examined, given their low potency and poor solubility. Overall, the data suggests that C-terminal lipidation can be better tolerated than lipidation on Lys, that a shorter lipid is better tolerated than a longer lipid, and that βAsp or γGlu spacers can alleviate the decrease in potency caused by longer lipids.

### Example 5

### Determination of the In Vivo Half-Life of Select Compounds

To examine the persistence of compounds in the blood circulation, an approach was devised to determine the concentration of compound in mouse blood by taking advantage of the ELISA measuring inhibition of EphA4-ephrinA5 interaction. The approach involves determining the concentration of compound remaining in the blood at various time points after administration by comparing the apparent IC₅₀ value for the compound remaining in plasma with the IC₅₀ value of a control obtained using mouse plasma spiked in vitro with a known compound concentration (corresponding to the theoretical concentration at the 0 time point, based on the amount of injected compound and a mouse blood volume of 2.5 mL) (FIG. 7). For example, the 5-fold increase in the apparent IC₅₀ value using plasma obtained 120 min after compound 14 administration indicates a 5-fold decrease in compound concentration in the blood, and therefore that 20% of the injected compound remains in the blood after 2 hours (Table 3).

Lipidated compounds dissolved in DMSO were diluted in 100 mL PBS (to a 5% final DMSO concentration) or in 100 mL PBS supplemented with 20 or 40 mg/mL BSA or 6.5 mM methyl-β-cyclodextrin for intravenous or intraperitoneal injection. Neutral pH of the compound solution was verified before injection. PEGylated compounds were dissolved in 250-300 mL PBS before intraperitoneal injection. Peptides were sterile filtered before administration. Blood from each mouse was collected at 3 different time points after compound injection, the first 2 times by retro-orbital bleeding and the third time by terminal cardiac bleeding under deep anesthesia. Red blood cells were removed by centrifugation from blood treated with heparin and the resulting plasma was frozen in aliquots.

To measure the amount of active APY-d3 present in the mouse blood after intravenous or intraperitoneal injection, the ELISA-based approach to measure compound inhibitory potency described above was used. For each time point, a dose-response curves by measuring inhibition of EphA4-ephrinA5 binding at different plasma dilutions was obtained. The concentration of active remaining in the blood circulation at various time points after administration was obtained by comparing the apparent IC₅₀ values for the compound recovered in the plasma with the IC₅₀ value of the same compound diluted in plasma in vitro at a concentration corresponding to the theoretical concentration in the mouse blood at the 0 time point, as shown in FIG. 7.

The in vivo half-life of representative lipidated compounds (FIGS. 3A, B, and C and Table 1) were examined. For compound 5 modified on Lys with octanoic acid (which is the shortest lipid used), only 5% of the compound remained in the blood 10 min after intravenous injection of the compound dissolved in PBS supplemented with 5% DMSO. However, the solubility of compound with longer lipids is poor in PBS containing 5% DMSO at the compound concentrations needed for administration. Therefore, a formulation was used including 20-40 mg/mL bovine serum albumin, which was sufficient to eliminate visible compound insoluble particles. This also has the advantage of avoiding rapid losses of injected lipidated compound that can occur before association with serum albumin in the mouse blood. For compound modified at the C-terminus with lauric acid, half-lives of 0.5-0.7 hours were observed for compound 12 and 14 injected intravenously and 0.5-1 hours for compound 11, 14 and 15 injected intraperitoneally. C-terminal attachment of the C14 lipid myristoyl acid through a γGluγGlu spacer in compound 16 prolongs to ˜2 hours the in vivo half-life of the compound after intraperitoneal injection. Formulation of compound 16 with methyl-β-cyclodextrin, a biocompatible cyclic oligosaccharide used to improve the solubility and bioavailability of hydrophobic drug, yielded similar in vivo half-life as the formulation with serum albumin. Of note, methyl-β-cyclodextrin does not affect the inhibitory potency of compound 16 (FIG. 6B). Finally, compound 18 and 19 with C-terminal palmitic acid had a half-life of ˜0.8 hours after intravenous injection and 1 to 1.5 hours after intraperitoneal injection.

Overall, considering potency and in vivo half-life, the lauroyl compound 15 and the myristoyl compound 16 are promising. These compounds, with IC₅₀ values of ˜50 and ˜80 nM, respectively, and a half-life in the circulation of 1 to 2 hours, could be injected intraperitoneally once or twice a day in mice to maintain a therapeutically effective blood concentration (0.5-1 μM (1)) or less frequently to achieve high but transient levels in the blood, which could be desirable in some cases.

### Example 6

### Evaluation of Compound 21

As an alternative strategy to promote complexation with serum albumin, compound 21 was prepared which includes the albumin-binding cyclic peptide SA21 (RLIEDICLPRWGCLWEDD (SEQ ID NO: 25), where the two underlined Cys residues form a disulfide bond. The SA21 peptide has been reported to bind albumin with higher affinity than lipids (Kd 470 nM for human serum albumin). Compound 21 appears to be as potent as APY-d3 (FIG. 2A-C and Table 1). However, the potency was greatly (˜8-fold) decreased in the presence of 20 mg/mL bovine serum albumin (FIG. 6D), suggesting that serum albumin binding to the SA21 moiety strongly interferes with the binding of the APY-d3 moiety to EphA4. In addition, it was found that the in vivo half-life of compound 21 (˜40 min) is shorter than that of the best lipidated compounds (FIG. 3B and C). This could be due to the presence of an arginine at the N-terminus of SA21, which can be susceptible to proteolytic cleavage separating the APY-d3 and SA21 moieties of compound 21, leading to the rapid excretion of the released APY-d3.

### Example 7

### Evaluation of Compounds APY-d3-PEG1, APY-d3-PEG2, APY-d3-PEG3 and APY-d3-PEG4

Compounds with half-lives longer than those of the lipidated compounds would allow a lower frequency of administration and thus would be more desirable in cases where persistent EphA4 inhibition is needed. Compounds APY-d3-PEG1, APY-d3-PEG2, APY-d3-PEG3 and APY-d3-PEG4 where a 30 kDa PEG was conjugated to the C-terminus of APY-d3 using an amide formation reaction and two alkane linkers of different reactivity to afford APY-d3-PEG1 and APY-d3-PEG2 (FIG. 4B and C). The PEG conjugate with the longer linker (APY-d3-PEG2) is the more potent of the two (FIG. 5A and Table 1), but with an IC₅₀ of ˜80 nM it is several fold less potent than non-PEGylated APY-d3. However, it was encouraging to find that the in vivo half-life of APY-d3-PEG2 is longer than 6 hours (FIG. 3D).

The approach used to generate APY-d3-PEG1 and APY-d3-PEG2, which involves amide formation, requires deprotection of the APY-d3 N-terminus after PEG conjugation. The late stage deprotection, can present issues in the purity of the final conjugate. Alternative strategies were developed for PEG conjugation, both of which can be carried out with a deprotected N-terminus thereby facilitating the synthesis of large amounts of PEGylated compounds. The first strategy involves conjugating the PEG to APY-d3 through a stable oxime linkage (FIG. 4D; APY-d3-PEG3). The aldehyde and aminooxy bioconjugation groups are known to react quantitatively and because the PEG conjugation is the final step in the synthesis, an acceptable level of purity is obtained. In addition, with this strategy the majority of the synthetic steps occur on the solid phase. This eliminates the need for repeated intermediate HPLC purifications, resulting in increased yields. The second strategy involves conjugating APY-d3 to 30 kDa PEG through a linker containing 3 amino-hexanoic acid groups and propargyl-glycine using click chemistry (FIG. 4E; APY-d3-PEG4). This conjugation can be carried out in solution, also with an unprotected N-terminus.

Unexpectedly, both APY-d3-PEG3 and APY-d3-PEG4 have similar potency as the non-PEGylated APY-d3 in ELISAs measuring inhibition of EphA4 ephrin-A5 interaction (FIG. 5A and Table 1). This is an improvement over the previously used PEGylation approach, which resulted in a several fold loss in potency. Most importantly, the in vivo half-lives of active APY-d3-PEG3 and APY-d3-PEG4 after intraperitoneal injection are ≥11 hours (FIG. 3D). Thus, APY-d3-PEG3 and APY-d3-PEG4 have superior characteristics compared to the best lipidated compounds when prolonged EphA4 inhibition is desired. Given the variability observed in the potency of some batches of APY-d3-PEG3, APY-d3-PEG4 was selected for experiments to measure inhibition of ligand-induced EphA4 activation in cells.

It was confirmed that APY-d3-PEG4, even at high concentrations, selectively inhibits only EphA4 among the Eph receptors, as is the case for APY-d3 (FIG. 5B) and found that APY-d3-PEG4 inhibits EphA4 tyrosine phosphorylation (indicative of activation) in cells stimulated with ephrin-A5. The inhibitory potency of APY-d3-PEG4 is 2-3 fold lower than that of APY-d3 (IC₅₀ values of ˜700 nM for APY-d3-PEG4 and ˜300 nM for APY-d3; FIG. 5C). Nevertheless, intraperitoneal administration of ˜0.8 mg/mouse APY-d3-PEG4 results in an early blood concentration of ˜9 μM, which decreases to ˜3 μM after 20 hours and to ˜0.4 μM after 48 hours. Since 0.5 to 1 μM APY-d3-PEG4 is sufficient to substantially inhibit EphA4 tyrosine phosphorylation induced by ephrinA5-Fc stimulation in cells, these data suggest that APY-d3-PEG4 can be administered intraperitoneally every other day in order to maintain a concentration sufficient to inhibit EphA4 in vivo. It should also be noted that compounds that have left the circulation to penetrate into tissues, such as tumor tissue, can exhibit an even lower rate of clearance.

### Example 8

Evaluation of EphA4 Y596 phosphorylation (activation) in cells

Immortalized cells stably transfected to express FLAG-tagged EphA4 were cultured at 37° C. in Dulbecco's Modified Eagle Medium (DMEM; #10-013-CV, Corning/Thermo Fisher Scientific) containing 10% fetal bovine serum (FBS) with 1% antibiotic-antimycotic solution (#30-004-Cl, Corning). To assess inhibition of EphA4 activation by compounds in cells, the cells were plated in 48-well plates. Once they reached 60-70% confluence, the cells were starved for 1 hour in culture medium without FBS, incubated for 20 min with compound at different concentrations, and then treated for an additional 10 min with 0.5 μg/mL ephrinA5-Fc (#374-EA-200, R&D Systems/Bio-Techne) to activate EphA4 in the continued presence of the compounds. Cells were then rinsed once with ice-cold PBS containing Ca′ and Mg (#17-513F, Lonza Bioscience) and collected in 250 μL TX100 buffer (1% Triton X-100, 10% glycerol, 2 mM EDTA in PBS) containing phosphatase and protease inhibitors (#78443, Thermo Fisher Scientific). Lysates were incubated for 30 min on a rocking platform at 4° C., and centrifuged for 10 min at 16,000 g.

For ELISAs, high binding capacity polystyrene 96-well plates were coated overnight at 4° C. with 1 μg/mL goat anti-FLAG antibody (#F3165, Sigma-Aldrich) in 50 μL/well borate buffer (0.1 M boric acid, 0.1 M Na borate, pH 8.7). The plates were then washed 3 times with TBST, incubated for 1.5 hours with 200 μL/well 5 mg/ml BSA (#3116964001, Roche) in PBS, washed 3 times with TBST, incubated overnight at 4° C. with 200 μL cell lysate/well, washed 3 times with TBST, incubated for 1.5 hour at room temperature with 100 μL/well anti-EphA2 pY588 antibody (#12677S, Cell Signaling Technology, which recognizes the conserved EphA4 pY596) diluted 1:4,000 in TBST, washed 3 times with TBST, incubated for 1 hour at room temperature with 100 μL/well anti-rabbit HRP (#A16110, Life Technologies) diluted 1:1,000 in TBST, washed 3 times with TBST, and incubated with 100 μL/well tetramethylbenzidine (TMB) substrate solution (#PI34028, Thermo Fisher Scientific) for ˜10 min before addition of 100 μL/well stop solution (#DY994, R&D Systems). OD₄₅₀ was then measured.

TABLE 1
Potency and in vivo half-life of EphA4 Antagonists

| | | | IC₅₀ ± | |
|---|---|---|---|---|
| | | | SE (n)² | |
| | | | by | |
| | | IC₅₀ ± | ELISA | |
| | | SE (n)² | with | |
| | | by | HSA or | |
| | SEQ ID | ELISA | BSA | In vivo |
| EphA4 Antagonist | NO: | (nM) | (nM) | half-life |
| APYCVYRβASWSC-NH₂ | APY-d2 | 30 ± 2 | nd | |
| | (SEQ ID | (19) | | |
| | NO: 40) | | | |
| APYCVYKβASWSC-NH₂ | 1 | 67 ± 10 | 55 ± 13 | |
| | | (14) | (3) | |
| APYCVYRβASWSCGGKG-NH₂ | 2 | 36 ± 7 | 59 ± 12 | |
| | | (7) | (4) | |
| βAPYCVYβASWSC-NH₂ | APY-d3 | 20 ± 2 | 36 ± 6 | nd |
| | (SEQ ID | (58) | (5)⁴ | |
| | NO: 41) | | | |
| βAPYCVYKβASWSC-NH₂ | 3 | 70 = 17 | nd | nd |
| | | (5) | | |
| βAPYCVYK(octanoyl)βASWSC-NH₂ | 4 | 34 = 4 | 100 ± 30 | nd |
| | | (10) | (3)³ | |
| βAPYCVYK(octanoyl)βASWSCGGRG-NH₂ | 5 | 26 ± 5 | 75 ± 24 | <10 min |
| | | (8) | (3)³ | (IV) |
| βAPYCVYK(octanyl-G)βASWSC-NH₂ | 6 | 42 ± 8 | 130 ± 40 | nd |
| | | (8) | (3)³ | |
| βAPYCVYRβASWSCGGK(octanoyl)G-NH₂ | 7 | 34 ± 6 | 110 ± 30 | nd |
| | | (11) | (5)³ | |
| βAPYCVYK(lauroyl)βASWSC-NH₂ | 8 | 390 ± 80 | nd | nd |
| | | (5) | | |
| βAPYCVYK(lauryl-G)βASWSC- NH₂ | 9 | 590 ± 70 | nd | nd |
| | | (5) | | |
| βAPYCVYK(lauroyl-βD)βASWSC-NH₂ | 10 | 110 ± 10 | nd | nd |
| | | (7) | | |
| βAPYCVYK(lauroyl-γE)βASWSC-NH₂ | 11 | 84 ± 13 | nd | ~40 min |
| | | (3) | | (IP) |
| βAPYCVYRβASWSCGGK(lauroyl)G-NH₂ | 12 | 240 ± 30 | 920 ± 180 | ~0.5 |
| | | (7) | (3)³ | hours |
| | | | | (IV) |
| βAPYCVYRβASWSCGGK(lauroyl-βD)G-NH₂ | 13 | 49 ± 11 | nd | nd |
| | | (3) | | |
| βAPYCVYRβASWSCGGK(lauroyl-γE)G-NH₂ | 14 | 52 ± 10 | nd | ~40 min |
| | | (4) | | (IV) |
| | | | | ~50 min |
| | | | | (IP) |
| βAPYCVYRβASWSCGGK(lauroyl-γEγE) | 15 | 25 ± 3 | 560 ± 120 | 40 min |
| G-NH₂ | | (9) | (4)³ | (IP) |
| βAPYCVYRβASWSCGGK(myristoyl-γEγE) | 16 | 77 ± 9 | 1,370 ± | ~100 |
| G-NH₂ | | (8) | 340 (3)³ | min (IP) |
| βAPYCVYK(palmitoyl-γE)BASWSC-NH2 | 17 | 2,300 ± | nd | nd |
| | | 190 (3) | | |
| βAPYCVYRβASWSCGGK(palmitoyl-γE) | 18 | 520 ± 80 | nd | ~50 min |
| G-NH₂ | | (4) | | (IV) |
| | | | | ~100 |
| | | | | min (IP) |
| βAPYCVYRβASWSCGGK(palmitoyl-γEγE) | 19 | 160 ± 30 | nd | ~70 min |
| G-NH₂ | | | | |
| | | (5) | | (IP) |
| βAPYCVYRβASWSCGGGSK(palmitoyl-γEγE) | 20 | 190 ± 20 | nd | nd |
| G-NH₂ | | (3) | | |
| βAPYCVYRβASWSCRLIEDICLPRWGCLWEDD- | 21 | 18 ± 4 | 130 ± 10 | ~40 min |
| NH₂ | | (5) | (5)⁴ | (IP) |
| βAPYCVYRβASWSC-linker1-PEG | APY- | 180 ± 10 | nd | nd |
| | d3- | (4) | | |
| | PEG1 | | | |
| | (SEQ ID | | | |
| | NO: 42) | | | |
| βAPYCVYRβASWSC-linker2-PEG | APY- | 83 ± 7 | nd | .6 hours |
| | d3- | (8) | | (IP) |
| | PEG2 | | | |
| | (SEQ ID | | | |
| | NO: 43) | | | |
| βAPYCVYRβASWSC-linker3-PEG | APY- | 15 ± 2 | nd | ~14 hours |
| | d3- | (8) | | (IP) |
| | PEG3 | | | |
| | (SEQ ID | | | |
| | NO: 44) | | | |
| βAPYCVYRβASWSC-linker4-PEG | APY- | 20 ± 3 | nd | ~11 hours |
| | d3- | (7) | | (IP) |
| | PEG4 | | | |
| | (SEQ ID | | | |
| | NO: 45) | | | |

², IC₅₀ values are averages of the IC₅₀ values from individual experiments, each with triplicate measurements; n = number of experiments. This table shows averages of all the experiments, whereas the panels in FIGS. 1, 2, 5 and 6 show comparisons from measurements carried out in the same experiments.
³, 40 mg/mL delipidatated HSA (human serum albumin).
⁴, 20 mg/ml BSA.
⁵, IV, intravenous administration; IP, intraperitoneal administration.

TABLE 2
Stability of EphA4 Antagonists

| | | IC₅₀ by | t1/2 in | t1/2 in |
|---|---|---|---|---|
| | | ELISA | plasma | mouse |
| EphA4 Antagonist | ID NO: | (nM) | In vivo | circulation |
| βAPYCVYRβASWSC-NH₂ | APY-d3 | 20 | >72 | <5 min |
| | (SEQ ID | | hours | |
| | NO: 41) | | | |
| βAPYCVYRβASWSCGGK | 15 | 25 | nd | ~40 min |
| (lauroyl-γEγE)G-NH₂ | | | | |
| βAPYCVYRβASWSC- | APY-d3- | 20 | nd | ~11 hours |
| linker4-PEG | PEG4 (SEQ | | | |
| | ID NO: 45) | | | |

TABLE 3

| | | FOLD | | |
|---|---|---|---|---|
| | | INCREASE IN | % PEPTIDE 14 | |
| | | APPARENTIC | REMAINING | |
| | Apparent | IC COMPARED | IN THE | ESTIMATED/ |
| Minutes | IC₅₀ (nM) | TO CONTROL | BLOOD | MEASURED |
| 0 | 81 | 1.0 | 100* | ESTIMATED |
| 60 | 168 | 2.1 | 48.2 | MEASURED |
| 120 | 371 | 4.6 | 21.8 | MEASURED |
| 240 | 1,820 | 22.5 | 4.5 | MEASURED |

### Example 9

### Therapeutic Usefulness of Compounds in ALS

To evaluate the therapeutic effects of compounds disclosed herein on ALS, EphA4 signaling inhibition will be examined using a mouse SOD1*G93A model of ALS. SOD1*G93A transgenic mice express human Cu/Zn superoxide dismutase 1 (SOD1) harboring a single amino acid substitution of glycine to alanine at codon 93. This pathogenic mutation is associated with early-onset familial ALS with hemizygotic SOD1*G93A animals exhibit neuronal degeneration due to progressive accumulation of detergent-resistant SOD-ubiquitin aggregates and aberrant neurofilament accumulations in degenerating motor neurons as well as reactive astroglia and microglia. The neuronal degeneration leads to limb grasping, widespread muscle weakness, atrophy and paralysis in one or more limbs due to loss of motor neurons from the spinal cord due to abnormal axonal transport. Transgenic mice also have an abbreviated life span.

Both SOD1*G93A mice, as well as non-transgenic mice used as age-matched controls, will be administered compounds disclosed herein, into the cerebral ventricles of the brain using a minipump. Behavioral analyses will reveal muscle function of SOD1*G93A mice compared to controls. Muscle and neuromuscular junction pathology of SOD1*G93A mice and controls then will be assayed using standard histological staining and immunohistochemistry using an amyloid beta (Aβ) antibody. These results will show that compounds disclosed herein will delay disease onset and pathogenesis, will decrease motor neuron loss, and/or will extend survival of the mice, thereby demonstrating the therapeutic effects in inhibiting EphA4 signaling and their usefulness in treating ALS. In addition, these data will confirm the findings obtained with the KYL peptide (a less potent EphA4 peptide antagonist) that inhibition of EphA4 signaling provides therapeutic benefits in Alzheimer's disease.

### Example 10

### Therapeutic Usefulness of Compounds in Alzheimer's Disease

To evaluate the therapeutic effects of compounds disclosed herein on Alzheimer's disease, EphA4 signaling inhibition will be examined using an APP/PS1 or other mouse model for Alzheimer's disease, including the TgCRND8 model encoding a double mutant form of amyloid precursor protein 695 (KM670/671NL+V717F) under the control of the PrP gene promoter. See, e.g., Chrishti, et al., Early-Onset Amyloid Deposition and Cognitive Deficits in Transgenic Mice Expressing a Double Mutant Form of Amyloid Precursor Protein 695, J. Biol. Chem 276 (24): 21562-21570 (2001), which is hereby incorporated by reference in its entirety. APP/PS1 double transgenic mice express a chimeric mouse/human amyloid precursor protein (Mo/HuAPP695swe) and a mutant human presenilin 1 (PS1-dE9) both directed to CNS neurons. Both pathogenic mutations are associated with early-onset Alzheimer's disease with transgenic mice showing visible β-amyloid plaque deposits in the brains by 6 to 7 months of age resulting in synaptic loss. APP/PS1 mice also exhibit certain behavioral abnormalities such as, impaired reversal learning of a food-rewarded four-arm spatial maze task, cognitive deficits in spatial learning and memory in the Morris water maze, and inhibition of hippocampal CA1 long-term potentiation (LTP). Thus, APP/PS1 mice demonstrate synaptic loss, reduced glutamatergic synaptic transmission and impaired synaptic plasticity in the hippocampus.

Both APP/PS1 mice, as well as non-transgenic mice used as age-matched controls, will be administered compounds disclosed herein, into the brain by intracerebral infusion for about 3 weeks. β-amyloid deposition and neuron loss in the cerebral cortex and hippocampus of APP/PS1 mice and controls then will be assayed using standard histological staining and immunohistochemistry using an amyloid beta (AB) antibody. These results will show that compounds disclosed herein will inhibit AB toxicity and/or will restore normal synaptic function and/or will restore LTP formation in APPP/PS1 mice, thereby demonstrating the therapeutic effects in inhibiting EphA4 signaling and their usefulness in treating Alzheimer's disease.

### Example 11

### Therapeutic Usefulness of Compounds in Stroke

To evaluate the therapeutic effects of compounds disclosed herein on stoke recovery, EphA4 signaling inhibition will be examined using a mouse photothrombosis model for stroke. See, e.g., Lemmens, et al., Modifying Expression Cof EphA4 and its Downstream Targets Improves Functional Recovery after Stroke, Hum. Mol. Genet. 22(11): 2214-2220 (2013), which is hereby incorporated by reference in its entirety. Focal cortical ischemia will be induced by photothrombosis in a wild-type strain of mice aged 3-4 months. Before the induction of photothrombosis, animals will receive training daily for 1 week on an accelerating rotarod treadmill (Ugo Basile), rotating from 4 to 40 r.p.m. over the course of 300 seconds in order to record three motor performance evaluations. The baseline performance will be recorded over six attempts the week after training. Induction of stroke will be evaluated 1 day after the procedure and animals will be excluded if the average and/or maximum performance over three attempts was 75% compared with baseline. Infarct volume will also be calculated using serial coronal sections immune-stained with antibodies against glial fibrillary acidic protein (GFAP) and compared to the contralateral side. Nerve regeneration will be evaluated by immunohistochemistry using antibodies against EphA4 and glial fibrillary acidic protein (GFAP).

Three days after induction of experimental stroke, mice will be divided into a treated group and an untreated group that will be used as age-matched controls. Treated mice will be administered compounds disclosed herein once daily for four weeks. Motor performance evaluations of treated and untreated animals will be conducted on post-stroke days 1, 7, 13, 19, 26, and 34. Infarct volume will also be measured.

These results will show that treatment with a compound disclosed herein will substantially improve motor function after experimental stroke. Mice treated with compounds disclosed herein will exhibit on improved rotarod performance relative to control animals (untreated) as well as increased axonal sprouting.

### Example 12

### Therapeutic Usefulness of Compounds in Nerve Regeneration

To evaluate the therapeutic effects of compounds disclosed herein on nerve regeneration, EphA4 signaling inhibition will be examined using a mouse corticospinal tract injury model for nerve regeneration. Spinal cord injury often leads to permanent incapacity because long axons cannot regenerate in the CNS. Eph receptors inhibit axon extension through an effect on the actin cytoskeleton. Severing of corticospinal axons causes EphA4 to accumulate at high levels in stumps of corticospinal axons, while a cognate ligand, ephrinB2, is upregulated at the lesion site so as to confine the injured axons.

Wild-type mice will be anesthetized and a spinal hemisection surgery will be performed in order to sever corticospinal axons in the T12-L1 region. Animals will be allowed to recover from the surgery and mice showing only complete paralysis will be used. Both hemisectioned mice, as well as un-operated mice used as age-matched controls, will be administered compounds disclosed herein, into the cervical spinal cord region by intracerebral infusion. Five weeks after spinal cord lesion, mice will be evaluated for nerve regeneration by using an anterograde tracing technique and immunohistochemistry using antibodies against EphA4 and glial fibrillary acidic protein (GFAP) as well as by using behavioral assessments before and after spinal hemisection like measuring stride length, ability to walk or climb on a grid and/or hindpaw grip strength.

These results will show that treatment with a compound disclosed herein will substantially improve recovery in hemisectioned animals relative to controls by promoting axon sprouting and/or improving limb function and recovery. Mice treated with a compound disclosed herein will exhibit axon sprouting, will show a reduction astrocytic gliosis and glial scaring, will demonstrate recovered stride length, the ability to walk on and/or climb a grid, and/or the ability to grasp with the affected hindpaw within 1-3 months of injury.

It is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

It is demonstrated that longer lipids such as myristoyl (14 carbons) and palmitoyl (16 carbons) most effectively increase half-life in the circulation. However, the longer lipids greatly decrease solubility, and thus more complex formulations are needed. More importantly, the longer lipids cause a greater decrease in compound inhibitory potency in ELISAs than shorter lipids. This was alleviated by using a γGluγGlu spacer to increase the distance between the lipid and the compound. Overall, the data suggests lauroyl compound 15 as the lipidated compound that best combines acceptable solubility, high potency (IC₅₀˜30 nM), and prolonged half-life in the circulation (˜40 min, based on measurement of the compound remaining in the blood at different times after intraperitoneal injection) (Table 3). A 1 hour half-life in the circulation is not ideal for studies in mouse disease models, although it should be noted that compound/drug in vivo half-lives are typically several times longer in the human blood circulation compared to the mouse. Furthermore, a shorter half-life can in some cases result in therapeutic benefit due to lower unwanted side effects.

It is also demonstrated that binding of serum albumin to the lipidated compounds and to the serum albumin binding moiety of compound 21 interferes with the ability to bind EphA4. In some cases, this could be an advantage because it would afford compound in an inactive state in the blood, thus avoiding potential unwanted side effects for example due to inhibition of EphA4 in T cells. Presumably, the compound would regain most of its activity once it reaches target tissues, where the concentration of serum albumin is much lower than in the blood. In tumors, where blood vessels can be leaky, lipidated APY-d3 could have an intermediate activity, which can vary depending on the serum albumin concentration in a particular tumor. Another potential advantage is that lipidation might promote compound penetration across the blood-brain barrier. This could be useful for the treatment of neurological disorders, in which the blood-brain barrier is often already partially compromised.

The effect of PEGylation was explored as another strategy to increase persistence in the mouse blood after intraperitoneal injection. A 30 kDa linear PEG was selected to improve APY-d3 in vivo half-life. PEG is a highly soluble, non-toxic and biologically inert material. PEGylation in APY-d3-PEG3 and APY-d3-PEG4 resulted in an in vivo half-life of more than 10 hours with minimal loss of EphA4 inhibitory activity.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only and is not intended to limit the scope of the present invention, which is defined solely by the claims. Accordingly, the present invention is not limited to that precisely as shown and described.

### EMBODIMENTS

Embodiment 1. A EphA4 receptor antagonist comprising a chemical entity having the structure of Formula I, βAPYCVYZ¹βASWSCZ² (SEQ ID NO: 22), or a pharmaceutically acceptable salt thereof, wherein Z¹ is R; Z² is L¹-PEG or GGK(lauroyl-γEγE)G-NH₂ (SEQ ID NO: 23); and L¹ is a linker.

Embodiment 2. The EphA4 receptor antagonist of embodiment 1, wherein L¹ is L^{1A}L^{1B}. L^{1A} is a peptide of at least two amino acids; L^{1B} is

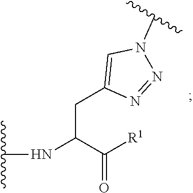

and R¹ is NH₂ or OH.

Embodiment 3. The EphA4 receptor antagonist of embodiment 2, wherein L^{1A} is a dipeptide, tripeptide, tetrapeptide, pentapeptide or hexapeptide.

Embodiment 4. The EphA4 receptor antagonist of embodiment 3, wherein L^{1A} is a dipeptide, tripeptide, or tetrapeptide.

Embodiment 5. The EphA4 receptor antagonist of embodiment 4, wherein L^{1A} is a dipeptide, or tripeptide.

Embodiment 6. The EphA4 receptor antagonist of embodiment 5, wherein LA is a tripeptide.

Embodiment 7. The EphA4 receptor antagonist of embodiment 5, wherein L^{1A} is AhxAhx.

Embodiment 8. The EphA4 receptor antagonist of embodiment 6, wherein L^{1A} is AhxAhxAhx.

Embodiment 9. The EphA4 receptor antagonist of any one of embodiments 1-8, wherein R¹ is NH₂.

Embodiment 10. The EphA4 receptor antagonist of any one of embodiments 1-7, wherein L¹ is

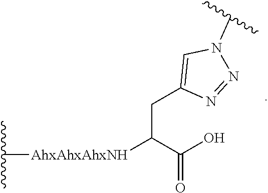

Embodiment 11. The EphA4 receptor antagonist of embodiment 2, wherein L^{1A} is a peptide of at least four amino acids; and the peptide does not include a C (Cys).

Embodiment 12. The EphA4 receptor antagonist of embodiment 11, wherein L^{1A} is a peptide of no greater than ten amino acids.

Embodiment 13. The EphA4 receptor antagonist according to embodiment 12, wherein L^{1A} is a tetrapeptide, pentapeptide or hexapeptide.

Embodiments 14. The EphA4 receptor antagonist according to embodiment 13, wherein L^{1A} is a tetrapeptide; and the tetrapeptide is GGKG (SEQ ID NO: 24).

Embodiments 15. The EphA4 receptor antagonist of any one of embodiments 1-14, wherein Z¹ is R; and PEG is OCH₂CH₂(OCH₂CH₂)ₛOH, OCH₂CH₂(OCH₂CH₂)ₛOCH₃ or OCH₂CH₂(OCH₂CH₂)ₛOCH₂CH₃; and s is 500 to 1000.

Embodiment 16. The EphA4 receptor antagonist of embodiment 1, wherein the chemical entity is compound APY-d3-PEG4, or a pharmaceutically acceptable salt thereof.

Embodiment 17. The EphA4 receptor antagonist of any one of embodiments 1-16, wherein the chemical entity has a cyclic structure and the two C (Cys) taken together have a disulfide bond.

Embodiments 18. The EphA4 receptor antagonist of any one of embodiments 1-17, wherein the EphA4 receptor antagonist has an equilibrium disassociation rate constant for an EphA4 receptor less than 500 nM, less than 450 nM, less than 400 nM, less than 350 nM, less than 300 nM, less than 250 nM, less than 200 nM, less than 150 nM, less than 100 nM, less than 75 nM, less than 50 nM, less than 25 nM, less than 20 nM, less than 15 nM, less than 10 nM, less than 5 nM, less than 1 nM, less than 0.5 nM or less than 0.1 nM.

Embodiment 19. The EphA4 receptor antagonist of any one of embodiments 1-18, wherein the EphA4 receptor antagonist has an equilibrium disassociation rate constant for an EphA4 receptor of between about 1 nM to about 10 nM, about 1 nM to about 25 nM, about 1 nM to about 75 nM, about 1 nM to about 100 nM, about 1 nM to about 125 nM, about 1 nM to about 150 nM, about 5 nM to about 10 nM, about 5 nM to about 25 nM, about 5 nM to about 75 nM, about 5 nM to about 100 nM, about 5 nM to about 125 nM, about 5 nM to about 150 nM, about 10 nM to about 25 nM, about 10 nM to about 50 nM, about 10 nM to about 75 nM, about 10 nM to about 100 nM, about 10 nM to about 125 nM, about 10 nM to about 150 nM, about 10 nM to about 175 nM or about 10 nM to about 200 nM.

Embodiment 20. The EphA4 receptor antagonist of any one of embodiments 1-19, wherein the EphA4 receptor antagonist has an equilibrium disassociation rate constant for an ephrin-binding pocket of an ephrin receptor other than an EphA4 receptor of at least 2-fold more, at least 3-fold more, at least 4-fold more, at least 5-fold more, at least 6-fold more, at least 7-fold more, at least 8-fold more, or at least 9-fold more, at least 10-fold more, at least 20-fold more, at least 30-fold more, at least 40-fold more, at least 50-fold more, at least 60-fold more, at least 70-fold more, at least 80-fold more, at least 90-fold more, at least 100-fold more, at least 200-fold more, at least 300-fold more, at least 400-fold more, at least 500-fold more, at least 600-fold more, at least 700-fold more, at least 800-fold more, at least 900-fold more, at least 1,000-fold more, at least 2,500-fold more, at least 5,000-fold more, at least 7,500-fold more or at least 10,000-fold more.

Embodiment 21. The EphA4 receptor antagonist of any one of embodiments 1-20, wherein the EphA4 receptor antagonist reduces EphA4 receptor activity by at least 10%, at least 20%, at least 30%, at least 40%, at least 50%, at least 60%, at least 70%, at least 80%, at least 60%, or at least 100%.

Embodiment 22. The EphA4 receptor antagonist of any one of embodiments 1-21, wherein the EphA4 receptor antagonist has a plasma half-life of at least 6 hours, at least 12 hours, at least 18 hours, at least 24 hours, at least 30 hours, at least 36 hours, at least 42 hours, at least 48 hours, at least 54 hours, at least 60 hours, at least 66 hours, at least 72 hours, at least 78 hours, at least 84 hours, at least 90 hours or at least 96 hours.

Embodiment 23. The EphA4 receptor antagonist according to any one of embodiments 1-22, wherein the modified EphA4 receptor antagonist has a plasma half-life of about 6 hours to about 24 hours, about 12 hours to about 36 hours, about 12 hours to about 48 hours, about 12 hours to about 60 hours, about 12 hours to about 72 hours, about 12 hours to about 84 hours, about 12 hours to about 96 hours, about 24 hours to about 36 hours, about 24 hours to about 48 hours, about 24 hours to about 60 hours, about 24 hours to about 72 hours, about 24 hours to about 84 hours, about 24 hours to about 96 hours, about 36 hours to about 48 hours, about 36 hours to about 60 hours, about 36 hours to about 72 hours, about 36 hours to about 84 hours, about 36 hours to about 96 hours, about 48 hours to about 60 hours, about 48 hours to about 72 hours, about 48 hours to about 84 hours, about 48 hours to about 96 hours, about 60 hours to about 72 hours, about 60 hours to about 84 hours, about 60 hours to about 96 hours, about 72 hours to about 84 hours, about 72 hours to about 96 hours or about 84 hours to about 96 hours.

Embodiment 24. A pharmaceutical composition comprising a EphA4 receptor antagonist of any one of embodiments 1-23.

Embodiment 25. The pharmaceutical composition according to embodiment 24, wherein the EphA4 receptor antagonist is present in an amount of between 95 ng to 1,005 μg.

Embodiment 26. The pharmaceutical composition of embodiment 24 or embodiment 25, wherein the pharmaceutical composition further comprises one or more pharmaceutical acceptable carriers.

Embodiment 27. Use of a EphA4 receptor antagonist of any one of embodiments 1-23 or a pharmaceutical composition of any one of embodiments 24-26 in the treatment of an EphA4-based disease, disorder or pathology.

Embodiment 28. Use of a EphA4 receptor antagonist of any one of embodiments 1-23 or a pharmaceutical composition of any one of embodiments 24-26 in the manufacture of a medicament for treating an EphA4-based disease, disorder or pathology.

Embodiment 29. A method of treating an EphA4-based disease, disorder or pathology, the method comprising administering a EphA4 receptor antagonist of any one of embodiments 1-23 or a pharmaceutical composition of any one of embodiments 24-26 to an individual in need thereof, wherein administration reduces one or more symptoms associated with the EphA4-based disease, disorder or pathology.

Embodiment 30. The use of embodiment 27 or embodiment 28 or the method of embodiment 29, wherein the EphA4-based disease, disorder or pathology comprises a condition, a disease, a disorder and/or pathology where a pathophysiology effect is due to dysregulation of EphA4 signaling in a manner that causes EphA4 signaling hyperactivity in cells or spatially or temporally aberrant EphA4 signaling.

Embodiment 31. The use or method of embodiment 30, wherein the EphA4-based disease, disorder or pathology is a neurodegenerative disease, a hearing loss, a promotion of nerve regeneration, a promotion of neuroprotection, or a cancer.

Embodiment 32. The use or method of embodiment 31, wherein the neurodegenerative disease is an Alexander disease, an Alper's disease, Alzheimer's disease, an amyotrophic lateral sclerosis, an ataxia telangiectasia, a Canavan disease, a Cockayne syndrome, a corticobasal degeneration, a Creutzfeldt-Jakob disease, a Guillain-Barre Syndrome a HIV-induced neurodegeneration, a Huntington disease, a Kennedy's disease, a Krabbe disease, a Lewy body dementia, a Machado-Joseph disease, a multiple sclerosis, a Parkinson's disease, a Pelizaeus-Merzbacher disease, a Pick's disease, a primary lateral sclerosis, a Refsum's disease, a Sandhoff disease, a Schilder's disease, a spinal cord injury, a Steele-Richardson-Olszewski disease, a stroke, a tabes dorsalis and/or a traumatic brain injury.

Embodiment 33. The method of any one of embodiment 29-32, wherein the one or more symptoms include abnormal movement, abnormal sensation, limb grasping, muscle weakness, atrophy, paralysis, abnormal inhibition of axon growth, abnormal axonal transport, aberrant synaptic function, synaptic transmission loss, impaired synaptic plasticity, synaptic loss, neuronal degeneration, motor neuron degeneration, motor neuron loss, poor neuronal survival, memory loss, impaired learning, dementia, β-amyloid plaque deposits, aberrant neurofilament accumulation, reactive astroglia and/or reactive microglia.

Embodiment 34. The use or method of embodiment 31, wherein the cancer is a glioblastoma, a gastric cancer, a pancreatic cancer, a prostate cancer, a breast cancer, a liver cancer, a leukemia, a melanoma, a lung cancer or a Sezary syndrome.

1.-38. (canceled)

39. A EphA4 receptor antagonist comprising a chemical entity having the structure of Formula I, βAPYCVYZ¹βASWSCZ², or a pharmaceutically acceptable salt thereof, wherein Z¹ is R; Z² is L¹-PEG; and L¹ is a linker.

40. The EphA4 receptor antagonist of claim 39, wherein L¹ is L^{1A}L^{1B}, L^{1A} is a peptide of at least two amino acids; L^{1B} is 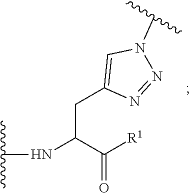
and R¹ is NH₂ or OH.

41. The EphA4 receptor antagonist of claim 40, wherein L^{1A} is a dipeptide, tripeptide, tetrapeptide, pentapeptide or hexapeptide.

42. The EphA4 receptor antagonist of claim 41, wherein L^{1A} is a dipeptide, tripeptide, or tetrapeptide.

43. The EphA4 receptor antagonist of claim 42, wherein L^{1A} is a dipeptide, or tripeptide.

44. The EphA4 receptor antagonist of claim 43, wherein L^{1A} is a tripeptide.

45. The EphA4 receptor antagonist of claim 43, wherein L^{1A} is AhxAhx.

46. The EphA4 receptor antagonist of claim 44, wherein L^{1A} is AhxAhxAhx.

47. The EphA4 receptor antagonist of claim 40, wherein R¹ is NH₂

48. The EphA4 receptor antagonist of claim 40, wherein L¹ is

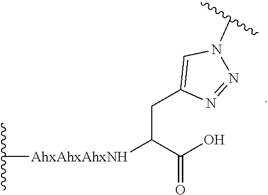

49. The EphA4 receptor antagonist of claim 40, wherein L^{1A} is a peptide of at least four amino acids; and the peptide does not include a C (Cys).

50. The EphA4 receptor antagonist of claim 49, wherein L^{1A} is a tetrapeptide, pentapeptide or hexapeptide.

51. The EphA4 receptor antagonist of claim 50, wherein L^{1A} is a tetrapeptide; and the tetrapeptide is GGKG.

52. The EphA4 receptor antagonist of claim 39, wherein PEG is OCH₂CH₂(OCH₂CH₂)ₛOH, OCH₂CH₂(OCH₂CH₂)ₛOCH₃ or OCH₂CH₂(OCH₂CH₂)ₛOCH₂CH₃; and s is 500 to 1000.

53. The EphA4 receptor antagonist of claim 39, wherein the chemical entity is compound APY-d3-PEG4, or a pharmaceutically acceptable salt thereof.

54. The EphA4 receptor antagonist of claim 39, wherein the EphA4 receptor antagonist has an equilibrium disassociation rate constant for an EphA4 receptor of between 1 nM to 10 nM, 1 nM to 25 nM, 1 nM to 75 nM, 1 nM to 100 nM, 1 nM to 125 nM, 1 nM to 150 nM, 5 nM to 10 nM, 5 nM to 25 nM, 5 nM to 75 nM, 5 nM to 100 nM, 5 nM to 125 nM, 5 nM to 150 nM, 10 nM to 25 nM, 10 nM to 50 nM, 10 nM to 75 nM, 10 nM to 100 nM, 10 nM to 125 nM, 10 nM to 150 nM, 10 nM to 175 nM or 10 nM to 200 nM.

55. The EphA4 receptor antagonist according to claim 39, wherein the modified EphA4 receptor antagonist has a plasma half-life in a range between 6 hours to 24 hours, 12 hours to 36 hours, 12 hours to 48 hours, 12 hours to 60 hours, 12 hours to 72 hours, 12 hours to 84 hours, 12 hours to 96 hours, 24 hours to 36 hours, 24 hours to 48 hours, 24 hours to 60 hours, 24 hours to 72 hours, 24 hours to 84 hours, 24 hours to 96 hours, 36 hours to 48 hours, 36 hours to 60 hours, 36 hours to 72 hours, 36 hours to 84 hours, 36 hours to 96 hours, 48 hours to 60 hours, 48 hours to 72 hours, 48 hours to 84 hours, 48 hours to 96 hours, 60 hours to 72 hours, 60 hours to 84 hours, 60 hours to 96 hours, 72 hours to 84 hours, 72 hours to 96 hours or 84 hours to 96 hours.

56. A pharmaceutical composition comprising the EphA4 receptor antagonist of claim 39; and one or more pharmaceutical acceptable carriers.

57. A method of treating an EphA4-based disease, disorder or pathology, the method comprising administering the EphA4 receptor antagonist of claim 39 to an individual in need thereof, wherein administration reduces one or more symptoms associated with the EphA4-based disease, disorder or pathology.

58. The method of claim 57, wherein the EphA4-based disease, disorder or pathology comprises a condition, a disease, a disorder and/or pathology where a pathophysiology effect is due to dysregulation of EphA4 signaling in a manner that causes EphA4 signaling hyperactivity in cells or spatially or temporally aberrant EphA4 signaling, wherein the EphA4-based disease, disorder or pathology is a neurodegenerative disease, a hearing loss, a promotion of nerve regeneration, a promotion of neuroprotection, or a cancer.


SEQUENCE LISTING (46 sequences)

SEQ ID NO: 1 - protein, 12 aa, synthetic construct
  SITE 8: note = Beta-alanine
  SITE 12: note = Amidated residue
APYCVYKXSWSC

SEQ ID NO: 2 - protein, 16 aa, synthetic construct
  SITE 8: note = Beta-alanine
  SITE 16: note = Amidated residue
APYCVYRXSWSCGGKG

SEQ ID NO: 3 - protein, 12 aa, synthetic construct
  SITE 1: note = Beta-alanine
  SITE 8: note = Beta-alanine
  SITE 12: note = Amidated residue
XPYCVYKXSWSC

SEQ ID NO: 4 - protein, 12 aa, synthetic construct
  SITE 1: note = Beta-alanine
  SITE 7: note = Octanoyl modified residue
  SITE 8: note = Beta-alanine
  SITE 12: note = Amidated residue
XPYCVYKXSWSC

SEQ ID NO: 5 - protein, 16 aa, synthetic construct
  SITE 1: note = Beta-alanine
  SITE 7: note = Octanoyl modified residue
  SITE 8: note = Beta-alanine
  SITE 16: note = Amidated residue
XPYCVYKXSWSCGGRG

SEQ ID NO: 6 - protein, 12 aa, synthetic construct
  SITE 1: note = Beta-alanine
  SITE 7: note = Octanyl-G modified residue
  SITE 8: note = Beta-alanine
  SITE 12: note = Amidated residue
XPYCVYKXSWSC

SEQ ID NO: 7 - protein, 16 aa, synthetic construct
  SITE 1: note = Beta-alanine
  SITE 8: note = Beta-alanine
  SITE 15: note = Octanoyl modified residue
  SITE 16: note = Amidated residue
XPYCVYRXSWSCGGKG

SEQ ID NO: 8 - protein, 12 aa, synthetic construct
  SITE 1: note = Beta-alanine
  SITE 7: note = Lauroyl modified residue
  SITE 8: note = Beta-alanine
  SITE 12: note = Amidated residue
XPYCVYKXSWSC

SEQ ID NO: 9 - protein, 12 aa, synthetic construct
  SITE 1: note = Beta-alanine
  SITE 7: note = Lauryl-G modified residue
  SITE 8: note = Beta-alanine
  SITE 12: note = Amidated residue
XPYCVYKXSWSC

SEQ ID NO: 10 - protein, 12 aa, synthetic construct
  SITE 1: note = Beta-alanine
  SITE 7: note = Lauroyl-beta-aspartic acid modified residue
  SITE 8: note = Beta-alanine
  SITE 12: note = Amidated residue
XPYCVYKXSWSC

SEQ ID NO: 11 - protein, 12 aa, synthetic construct
  SITE 1: note = Beta-alanine
  SITE 7: note = Lauroyl-beta-gamma-glutamic acid modified residue
  SITE 8: note = Beta-alanine
  SITE 12: note = Amidated residue
XPYCVYKXSWSC

SEQ ID NO: 12 - protein, 16 aa, synthetic construct
  SITE 1: note = Beta-alanine
  SITE 8: note = Beta-alanine
  SITE 15: note = Lauroyl modified residue
  SITE 16: note = Amidated residue
XPYCVYRXSWSCGGKG

SEQ ID NO: 13 - protein, 16 aa, synthetic construct
  SITE 1: note = Beta-alanine
  SITE 8: note = Beta-alanine
  SITE 15: note = Lauroyl-beta-aspartic acid modified residue
  SITE 16: note = Amidated residue
XPYCVYRXSWSCGGKG

SEQ ID NO: 14 - protein, 16 aa, synthetic construct
  SITE 1: note = Beta-alanine
  SITE 8: note = Beta-alanine
  SITE 15: note = Lauroyl-beta-gamma-glutamic acid modified residue
  SITE 16: note = Amidated residue
XPYCVYRXSWSCGGKG

SEQ ID NO: 15 - protein, 16 aa, synthetic construct
  SITE 1: note = Beta-alanine
  SITE 8: note = Beta-alanine
  SITE 15: note = Lauroyl-gamma-glutamic acid-gamma glutamic acid modified residue
  SITE 16: note = Amidated residue
XPYCVYRXSWSCGGKG

SEQ ID NO: 16 - protein, 16 aa, synthetic construct
  SITE 1: note = Beta-alanine
  SITE 8: note = Beta-alanine
  SITE 15: note = Myristoyl-gamma-glutamic acid-gamma glutamic acid modified residue
  SITE 16: note = Amidated residue
XPYCVYRXSWSCGGKG

SEQ ID NO: 17 - protein, 12 aa, synthetic construct
  SITE 1: note = Beta-alanine
  SITE 7: note = Palmitoyl-gamma-glutamic acid modified residue
  SITE 8: note = Beta-alanine
  SITE 12: note = Amidated residue
XPYCVYKXSWSC

SEQ ID NO: 18 - protein, 16 aa, synthetic construct
  SITE 1: note = Beta-alanine
  SITE 8: note = Beta-alanine
  SITE 15: note = Palmitoyl-gamma-glutamic acid modified residue
  SITE 16: note = Amidated residue
XPYCVYRXSWSCGGKG

SEQ ID NO: 19 - protein, 16 aa, synthetic construct
  SITE 1: note = Beta-alanine
  SITE 8: note = Beta-alanine
  SITE 15: note = Palmitoyl-gamma-glutamic acid-gamma glutamic acid modified residue
  SITE 16: note = Amidated residue
XPYCVYRXSWSCGGKG

SEQ ID NO: 20 - protein, 18 aa, synthetic construct
  SITE 1: note = Beta-alanine
  SITE 8: note = Beta-alanine
  SITE 17: note = Palmitoyl-gamma-glutamic acid-gamma glutamic acid modified residue
  SITE 18: note = Amidated residue
XPYCVYRXSWSCGGGSKG

SEQ ID NO: 21 - protein, 30 aa, synthetic construct
  SITE 1: note = Beta-alanine
  SITE 8: note = Beta-alanine
  SITE 30: note = Amidated residue
XPYCVYRXSWSCRLIEDICLPRWGCLWEDD

SEQ ID NO: 22 - protein, 16 aa, synthetic construct
  SITE 1: note = Beta-alanine
  SITE 8: note = Beta-alanine
  SITE 15: note = Lauroyl-gamma-glutamic acid-gamma glutamic acid modified residue
  SITE 16: note = Amidated residue
  VARIANT 13..16: note = This region may be absent and if absent, position 12 is attached to polyethylene glycol via a linker
XPYCVYRXSWSCGGKG

SEQ ID NO: 23 - protein, 4 aa, synthetic construct
  SITE 3: note = Lauroyl-gamma-glutamic acid-gamma glutamic acid modified residue
  SITE 4: note = Amidated residue
GGKG

SEQ ID NO: 24 - protein, 4 aa, synthetic construct
GGKG

SEQ ID NO: 25 - protein, 18 aa, synthetic construct
  SITE 7..13: note = Intrachain disulfide bond
RLIEDICLPRWGCLWEDD

SEQ ID NO: 26 - protein, 30 aa, synthetic construct
  SITE 1: note = Beta-alanine
  VARIANT 7: note = R, K, octanoyl modified K, lauroyl modified K, lauryl-G modified K, lauroyl-beta-aspartic acid modified K, lauroyl-gamma-glutamic acid modified K, palmitoyl-gamma-glutamic acid, or absent
  SITE 8: note = Beta-alanine
  VARIANT 13..30: note = This region may be absent or encompass one the following: amide, GGRG-amide, GGK(side chain modified with lauroyl)G-amide, GGK(side chain modified with lauroyl-beta-aspartic acid)G-amide, GGK(side chain modified with lauroyl-gamma-glutamic acid)G-amide, GGK(side chain modified with lauroyl-gamma-glutamic acid-gamma-glutamic acid)G-amide, GGK(side chain modified with myristoyl-gamma-glutamic acid-gamma-glutamic acid)G-amide, GGK(side chain modified with palmitoyl-gamma-glutamic acid)G-amide, GGK(side chain modified with palmitoyl-gamma-glutamic acid-gamma-glutamic acid)G-amide, GGGSK(side chain modified with palmitoyl-gamma-glutamic acid-gamma-glutamic acid)G-amide, RLIEDICLPRWGCLWEDD-amide, or polyethylene glycol attached via a linker
XPYCVYXXSWSCXXXXXXXXXXXXXXXXXX

SEQ ID NO: 27 - protein, 4 aa, synthetic construct
  SITE 4: note = Amidated residue
GGRG

SEQ ID NO: 28 - protein, 4 aa, synthetic construct
  SITE 3: note = Lauroyl modified residue
  SITE 4: note = Amidated residue
GGKG

SEQ ID NO: 29 - protein, 4 aa, synthetic construct
  SITE 3: note = Lauroyl-beta-aspartic acid modified residue
  SITE 4: note = Amidated residue
GGKG

SEQ ID NO: 30 - protein, 4 aa, synthetic construct
  SITE 3: note = Lauroyl-beta-gamma-glutamic acid modified residue
  SITE 4: note = Amidated residue
GGKG

SEQ ID NO: 31 - protein, 4 aa, synthetic construct
  SITE 3: note = Myristoyl-gamma-glutamic acid-gamma glutamic acid modified residue
  SITE 4: note = Amidated residue
GGKG

SEQ ID NO: 32 - protein, 4 aa, synthetic construct
  SITE 3: note = Palmitoyl-gamma-glutamic acid modified residue
  SITE 4: note = Amidated residue
GGKG

SEQ ID NO: 33 - protein, 4 aa, synthetic construct
  SITE 3: note = Palmitoyl-gamma-glutamic acid-gamma glutamic acid modified residue
  SITE 4: note = Amidated residue
GGKG

SEQ ID NO: 34 - protein, 6 aa, synthetic construct
  SITE 5: note = Palmitoyl-gamma-glutamic acid-gamma glutamic acid modified residue
  SITE 6: note = Amidated residue
GGGSKG

SEQ ID NO: 35 - protein, 18 aa, synthetic construct
  SITE 18: note = Amidated residue
RLIEDICLPRWGCLWEDD

SEQ ID NO: 36 - protein, 15 aa, synthetic construct
  SITE 1: note = Fluorenylmethoxycarbonyl-Beta-alanine
  SITE 8: note = Beta-alanine
  SITE 13: note = Beta-alanine
  SITE 14: note = Beta-alanine
  SITE 15: note = Amidated residue
XPYCVYRXSWSCXXK

SEQ ID NO: 37 - protein, 16 aa, synthetic construct
  SITE 1: note = Beta-alanine
  SITE 8: note = Beta-alanine
  SITE 15: note = Lauroyl-gamma-glutamic acid-gamma glutamic acid modified residue
  SITE 16: note = Amidated residue
  SITE 4..12: note = Intrachain disulfide bond
XPYCVYRXSWSCGGKG

SEQ ID NO: 38 - protein, 16 aa, synthetic construct
  SITE 1: note = Beta-alanine
  SITE 8: note = Beta-alanine
  SITE 13: note = Aminohexanoic acid
  SITE 14: note = Aminohexanoic acid
  SITE 15: note = Aminohexanoic acid
  SITE 16: note = Propargylglycine modifed with polyethylene glycol 30 monomethyl ether-triazole
  SITE 4..12: note = Intrachain disulfide bond
XPYCVYRXSWSCXXXX

SEQ ID NO: 39 - protein, 16 aa, synthetic construct
  SITE 1: note = Beta-alanine
  SITE 3: note = Tert-butyl modified residue
  SITE 4: note = Trityl modified residue
  SITE 6: note = Tert-butyl modified residue
  SITE 7: note = 2,2,4,6,7-Pentamethyldihydrobenzofuran-5-sulfonyl modified residue
  SITE 8: note = Beta-alanine
  SITE 9: note = Tert-butyl modified residue
  SITE 10: note = Tert-Butoxycarbonyl modified residue
  SITE 11: note = Tert-butyl modified residue
  SITE 12: note = Trityl modified residue
  SITE 13: note = Aminohexanoic acid
  SITE 14: note = Aminohexanoic acid
  SITE 15: note = Aminohexanoic acid
  SITE 16: note = Propargylglycine
XPYCVYRXSWSCXXXX

SEQ ID NO: 40 - protein, 12 aa, synthetic construct
  SITE 8: note = Beta-alanine
  SITE 12: note = Amidated residue
APYCVYRXSWSC

SEQ ID NO: 41 - protein, 12 aa, synthetic construct
  SITE 1: note = Beta-alanine
  SITE 8: note = Beta-alanine
  SITE 12: note = Amidated residue
XPYCVYRXSWSC

SEQ ID NO: 42 - protein, 15 aa, synthetic construct
  SITE 1: note = Beta-alanine
  SITE 8: note = Beta-alanine
  SITE 13: note = Beta-alanine
  SITE 14: note = Beta-alanine
  SITE 15: note = Amidated residue sidechain modified with polyethylene glycol monomethyl ether containing moiety
XPYCVYRXSWSCXXK

SEQ ID NO: 43 - protein, 15 aa, synthetic construct
  SITE 1: note = Beta-alanine
  SITE 8: note = Beta-alanine
  SITE 13: note = Beta-alanine
  SITE 14: note = Beta-alanine
  SITE 15: note = Amidated residue sidechain modified with polyethylene glycol monomethyl ether containing moiety
XPYCVYRXSWSCXXK

SEQ ID NO: 44 - protein, 14 aa, synthetic construct
  SITE 1: note = Beta-alanine
  SITE 8: note = Beta-alanine
  SITE 13: note = Beta-alanine
  SITE 14: note = Amidated residue sidechain modified with polyethylene glycol monomethyl ether containing moiety
XPYCVYRXSWSCXK

SEQ ID NO: 45 - protein, 15 aa, synthetic construct
  SITE 1: note = Beta-alanine
  SITE 8: note = Beta-alanine
  SITE 13: note = Aminohexanoic acid
  SITE 14: note = Aminohexanoic acid
  SITE 15: note = Aminohexanoic acid sidechain modified with polyethylene glycol monomethyl ether containing moiety
XPYCVYRXSWSCXXX

SEQ ID NO: 46 - protein, 4 aa, synthetic construct
GGRG